(19) United States
(12) Patent Application Publication
(10) Pub. No.: US 2015/0018486 A1
(43) Pub. Date: Jan. 15, 2015

(54) Nanotherapeutic Colloidal Metal Compositions and Methods

(71) Applicant: Cytlmmune Sciences, Inc., Rockville, MD (US)

(72) Inventors: Lawrence Tamarkin, Rockville, MD (US); Giulio F. Paciotti, Ellicott City, MD (US); Marja S. Huhta, Seattle, WA (US)

(73) Assignee: Cytlmmune Sciences, Inc., Rockville, MD (US)

(21) Appl. No.: 14/181,179

(22) Filed: Feb. 14, 2014

Related U.S. Application Data
(63) Continuation of application No. 12/843,504, filed on Jul. 26, 2010, now abandoned.
Application No. 12/843,504 is a continuation-in-part of application No. 12/235,342, filed on Sep. 22, 2008, now abandoned.
(60) Provisional application No. 60/974,310, filed on Sep. 21, 2007.
(60) Provisional application No. 60/981,920, filed on Oct. 23, 2007.
(60) Provisional application No. 61/069,108, filed on Mar. 12, 2008.
(60) Provisional application No. 61/069,905, filed on Mar. 19, 2008.
(60) Provisional application No. 61/040,022, filed on Mar. 27, 2008.
(60) Provisional application No. 61/124,290, filed on Apr. 15, 2008.
(60) Provisional application No. 61/126,899, filed on May 8, 2008.
(60) Provisional application No. 61/228,243, filed on Jul. 24, 2009.
(60) Provisional application No. 61/326,424, filed on Apr. 21, 2010.

(51) Int. Cl. A61K 38/19; A61K 47/48; A61K 51/06

(52) U.S. Cl. CPC A61K 38/191; A61K 51/06; A61K 47/48861; A61K 47/48215; A61K 47/48176
USPC 525/54.1; 530/351; 525/284; 525/329.4

(57) ABSTRACT

The present invention comprises compositions and methods for delivery systems of agents, including therapeutic compounds, pharmaceutical agents, drugs, detection agents, nucleic acid sequences and biological factors. In general, the nanotherapeutic compositions of the present invention comprise a platform comprising a colloidal metal, a targeting ligand such a tumor necrosis factor, a stealth agent such as polyethylene glycol, and one or more diagnostic or therapeutic agents for delivery. The invention also comprises methods and compositions for making such nanotherapeutic compositions and for the treatment of cancer.

### CROSS REFERENCE TO RELATED APPLICATIONS

This application is a continuation of U.S. patent application Ser. No. 12/843,504 filed on Jul. 26, 2010, which is a continuation-in-part of U.S. patent application Ser. No. 12/235,342, filed Sep. 22, 2008, which claims the benefit of U.S. Provisional Patent Application No. 60/974,310, filed Sep. 21, 2007, U.S. Provisional Patent Application No. 60/981,920, filed Oct. 23, 2007, U.S. Provisional Patent Application No. 61/069,108, filed Mar. 12, 2008, U.S. Provisional Patent Application No. 61/069,905, filed Mar. 19, 2008, U.S. Provisional Patent Application No. 61/040,022, filed Mar. 27, 2008, filed Apr. 11, 2008, U.S. Provisional Patent Application No. 61/124,290, filed Apr. 15, 2008, and U.S. Provisional Patent Application No. 61/126,899, filed May 8, 2008. This application also claims the benefit of U.S. Provisional Patent Application No. 61/228,243 filed Jul. 24, 2009 and U.S. Provisional Patent Application No. 61/326,424 filed Apr. 21, 2010.

### FIELD OF THE INVENTION

The present invention relates to compositions and methods for generalized delivery of agents and delivery of agents to specific sites. In general, the present invention relates to colloidal metal compositions and methods for making and using such compositions.

### BACKGROUND OF THE INVENTION

It has long been a goal of therapeutic treatment to find the magic bullet that would track to the site of need and deliver a therapeutic response without undue side effects. Many approaches have been tried to reach this goal. Therapeutic agents have been designed to take advantage of differences in active agents, such as hydrophobicity or hydrophilicity, or size of therapeutic particulates for differential treatment by cells of the body. Therapies exist that deliver therapeutic agents to specific segments of the body or to particular cells by in situ injection, and either use or overcome body defenses such as the blood-brain barrier, that limit the delivery of therapeutic agents.

One method that has been used to specifically target therapeutic agents to specific tissues or cells is delivery based on the combination of a therapeutic agent and a binding partner of a specific receptor. For example, the therapeutic agent may be cytotoxic or radioactive and when combined with a binding partner of a cellular receptor, cause cell death or interfere with genetic control of cellular activities once bound to the target cells. This type of delivery device requires having a receptor that is specific for the cell-type to be treated, an effective binding partner for the receptor, and an effective therapeutic agent. Molecular genetic manipulations have been used to overcome some of these problems.

Specific methods for delivering genetic sequences into exogenous cells or for overexpression of endogenous sequences, are of great interest at the current time. Various techniques for inserting genes into cells are used. These techniques include precipitation, viral vectors, direct insertion with micropipettes and gene guns, and exposure of nucleic acid to cells. A widely used precipitation technique uses calcium phosphate to precipitate DNA to form insoluble particles. The goal is for at least some of these particles to become internalized within the host cells by generalized cellular endocytosis. This results in the expression of the new or exogenous genes. This technique has a low efficiency of entry of exogenous genes into cells with the resulting expression of the genes. The internalization of the genes is non-specific with respect to which cells are transfected because all exposed cells are capable of internalizing the exogenous genes since there is no reliance upon any particular recognition site for the endocytosis. This technique is used widely in vitro, but because of the lack of specificity of target cell selection and poor uptake by highly differentiated cells, its use in vivo is not contemplated. In addition, its use in vivo is limited by the insoluble nature of the precipitated nucleic acids.

A similar technique involves the use of DEAE-Dextran for transfecting cells in vitro. DEAE-Dextran is deleterious to cells and also results in non-specific insertion of nucleic acids into cells. This method is not advisable in vivo.

Other techniques for transfecting cells, or providing for the entry of exogenous genes into cells are also limited. Using viruses as vectors has some applicability for in vitro and in vivo introduction of exogenous genes into cells. There is always the risk that the presence of viral proteins will produce unwanted effects in an in vivo use. Additionally, viral vectors may be limited as to the size of exogenous genetic material that can be ferried into the cells. Repeated use of viral vectors raises an immunological response in the recipient and limits the times the vector can be used.

Exogenous gene delivery has also been used with liposome-entrapped nucleic acids. Liposomes are membrane-enclosed sacs that can be filled with a variety of materials, including nucleic acids. Liposome delivery does not provide for uniform delivery to cells because of uneven filling of the liposomes. Though liposomes can be targeted to specific cellular types if binding partners for receptors are included, liposomes suffer from breakage problems, and thus delivery is not specific.

Brute force techniques for inserting exogenous nucleic acids include puncturing cellular membranes with micropipettes or gene guns to insert exogenous DNA into a cell. These techniques work well for some procedures, but are not widely applicable. They are highly labor intensive and require very skilled manipulation of the recipient cell. These are not techniques that are simple procedures that work well in vivo. Electroporation, using electrical methods to change the permeability of the cellular membrane, has been successful for some in vitro therapies for insertion of genes into cells.

There have been some attempts at targeted delivery of DNA for specific cells that relied upon the presence of receptors for glycoproteins. The delivery system used polycations, such as polylysine, that were noncovalently bonded to DNA, and that were also covalently bonded to a ligand. Such use of covalently bonding of the polycations to a ligand does not allow for the disassembly of the delivery system once the cellular internalization mechanisms begin. This large complex, covalently bonded delivery system is very unlike the way nucleic acids are naturally found within cells.

As is evident to those skilled in the art, there continues to be a long felt need for targeting cancer therapeutics to solid tumors facilitated by particle delivery systems capable of escaping phagocytic clearance by the reticuloendothelial system (RES; Papisov 1998, Moghimi, 1998 and Woodle, 1998). Under ideal conditions such delivery systems would preferentially extravasate the tumor vasculature and accumulate within the tumor microenvironment (Anaya's 1999, Maruyama 1999). In addition, a desirable particle delivery system capable of sequestering a cancer drug solely within a tumor would also reduce the accumulation of the drug in healthy organs (Papisov 1998, Moghimi, 1998 and Woodle, 1998, Nafayasu 1999, Maruyama 1999). Consequently, these delivery systems would increase the relative efficacy or safety of a cancer therapy, and thus would serve to increase the drug's therapeutic index.

The field of particle-based drug delivery is currently focused on two chemically distinct colloidal particles, liposomes and biodegradable polymers (Muller 2000, Jain, 1998, Rafferty, 1996, Ogawa 1997, and Maruyama, 1998). Both delivery systems encapsulate the active drug. The drug is released from the particle as it lyses, in the case of liposomes, or disintegrates as described for biodegradable polymers.

Colloidal gold nanoparticles represent a completely novel technology in the field of particle-based tumor targeted drug delivery. The synthesis of these particles was first reported by Michael Faraday, who, in 1857, described the chemical process for the production of nano-sized particles of gold from gold chloride and sodium citrate (Faraday, 1857). In the 1950's the discovery that these particles could bind protein biologics without altering their activity paved the way for their use in hand-held immunodiagnostics and in histopathology (Chandler, 2001). Of particular relevance is the use of radioactive colloidal gold nanoparticles, made from AU₁₉₈, for the treatment of liver cancer and sarcoma (Rubin 1964, Root 1954). Intravenous administration of these nanoparticles resulted in drug-associated toxicities due to radiation exposure. However, no demonstrable toxicities were noted from the particles themselves. More recently gold nanoparticles have been assembled into scaffolds for use in DNA diagnostics and biosensors (Mirkin 1996).

The emerging field of bionanotechnology (or nanobiotechnology) offers the potential for the development of exquisitely sensitive diagnostics and organ/tumor-targeted therapies. For example, the miniaturization of diagnostics may not only provide clinicians with a more complete snapshot of blood chemistries, hormones and growth factors in both normal and diseases states, but may also allow them to tract the efficacy of putative therapeutics [Koehne et al., 2004]. Complementing its diagnostic advances bionanotechnology also holds the promise of increasing the therapeutic index, a measure of the benefit/risk ratio, of current cancer therapies, as a prime example [Papisov et al., 1998, Moghimi et al., 1998, Woodle, 1998, Nafayasu et al., 1999, Maruyama et al., 1999]. Indeed, the blending of material science and tumor biology is leading to the development of innovative vectors with the potential of achieving the long-sought-after goal of tumor-targeted drug delivery, getting the active agent(s) solely where it's needed, at the solid tumor. Yet, to successfully achieve this goal, nanoparticle delivery systems must overcome the biological barriers that are naturally present in the body, as well as those that develop during tumor growth and progression. Such natural barriers include, but are not limited to, clearance by the reticuloendothelial system (by size or oposonization), tumor angiogenesis leading to an increase in interstitial (tumor) fluid pressure, ligand/receptor based nanotherapeutic targeting, barriers within the tumor interstitium: intra-tumor barriers established during the formation and cellular heterogeneity of solid tumors.

Although some advances have been made in addressing the problems of the natural barriers listed above, many challenges still remain. For example, tumor-targeting drug delivery vectors have not yet approached ‘true’ or optimal nanometer size, which will not only diminish the likelihood of their being opsonized in the blood and taken up by the reticuloendothelial system (RES; i.e., larger particles activate complement better than smaller particles) but also prevent their clearance in the narrow confines of the inter-endothelial slits present in the red-pulp of the spleen. It is thought that to further improve RES avoidance, hydrophilic polymers may be grafted onto the surface of currently available nanoparticle systems. Once these nanoparticle vectors are free to circulate throughout the body, it is thought that they may passively as well as actively sequester in and around a solid tumor due to the inherent leakiness of the tumor neovasculature and the presence of tumor-specific ligands on the surface of these nanoparticle vectors.

Those skilled in the art also realize a need for the last element in building effective nanotherapeutics that lies in the ability to develop vectors that effectively deliver multiple therapeutic agents to the heterogeneous populations of cancer cells comprising a solid tumor. In its simplest model a solid tumor may be viewed as an organ containing multiple cell types that act in concert to promote tumor growth (Spremulli and Dexter, 1983, Dexter et al., 1978]. Thus, drugs that target a single type of cell for therapeutic intervention may only provide marginal anti-tumor effect. Furthermore, in many cases solid tumor cells exhibit a continuum of phenotypes during disease progression and/or in response to therapy. Consequently it seems unlikely that single agent therapies, regardless of the ability of the nanoparticle delivery system to sequester them in the solid tumor, will prove effective against the myriad cells present within the malignancy. To overcome this limitation, what is needed therefore is a next generation nanotherapeutics that must not only find their way to the solid tumor but must also effectively destroy the diverse populations of cells promoting tumor growth.

Simple, efficient delivery systems for delivery of specific therapeutic agents to specific sites in the body for the treatment of diseases or pathologies or for the detection of such sites are not currently available. For example, current treatments for cancer include administration of chemotherapeutic agents and other biologically active factors such as cytokines and immune factors that impact the entire organism. The side effects include organ damage, loss of senses such as taste and feel, and hair loss. Such therapies provide treatment for the condition, but also require many adjunct therapies to treat the side effects.

What is needed are compositions and methods for delivery systems of agents that effect the desired cells or site. These delivery systems could be used for delivery to specific cells for agents of all types, including detection and therapeutic agents. What is also needed are delivery systems that do not cause unwanted side effects in the entire organism.

### SUMMARY OF INVENTION

The present invention comprises compositions and methods for delivery of agents, including, but not limited to, therapeutic compounds, pharmaceutical agents, drugs, prodrugs, detection agents, nucleic acid sequences and biological factors. In one exemplary embodiment, the present invention provides these delivery or vector compositions as multifunctional nanotherapeutics essentially comprising a nanoparticle platform for assembling, one or more stealth agents, one or more active agents and optionally one or more targeting ligands. In another exemplary embodiment one or more active agents, and optionally one or more targeting ligands, are assembled on polymer backbones to form functionalized polymers, wherein the functionalized polymers are connected to a nanoparticle platform. The present invention further comprises methods and compositions for making nanoparticle vector compositions.

The nanoparticle platforms of the present invention provide a base on which other molecules may be assembled to form the vector compositions of the present invention. The nanoparticle platforms may comprise materials such as, but not limited to, gold nanoparticles, silver nanoparticles, silica nanoparticles, iron nanoparticles, metal hybrid nanoparticles such as gold/iron nanoparticles, nanoshells, gold nanoshells, silver nanoshells, gold nanorods, silver nanorods, metal hybrid nanorods, quantum dots, nanoclusters, liposomes, dendrimers, metal-liposome particles, metal-dendrimer nanohybrids and carbon nanotubes. Attachment of the one or more active agents to a nanoparticle platform may slow the hydrolytic conversion of the active agents in the circulation. In addition, the nanoparticle platform may also facilitate the accumulation of the active agent at the site of disease (i.e. a solid tumor). In certain embodiments, the attachment of the active agents to the nanoparticle platform may also control the rate at which the agents are converted to their active from.

The active agents which can be delivered, both in vitro and in vivo, using the vector compositions of the present invention include both therapeutic and diagnostic agents. In one exemplary embodiment, the active agent is an anti-cancer therapeutic agent. In certain exemplary embodiments, the anti-cancer agent is a chemotherapeutic agent. In certain other exemplary embodiments, the chemotherapeutic agent is paclitaxel. In certain other exemplary embodiments, the therapeutic agent is a chelating agent for a therapeutic radionuclide, such as ⁹⁰Y. In another exemplary embodiment, the active agent comprises a combination of therapeutic agents. In certain exemplary embodiments, the therapeutic agents are tumor necrosis factor (TNF) and paclitaxel. In yet another exemplary embodiment, the active agent is a diagnostic agent. Suitable detection agents include, but are not limited to, magnetic, paramagnetic, radioactive, fluorescent, and chemiluminscent detection agents. In certain exemplary embodiments, the detection agent is a chelating agent for an imaging radionuclide, such as ^{99m}Tc. In yet another exemplary embodiment, the active agent is a combination of therapeutic and detection agents.

The vector compositions of the present invention may optionally contain a targeting molecule to facilitate site-specific delivery to a particular cell or tissue type. For instance, the targeting molecule may be used to deliver the vector composition and its therapeutic or diagnostic payload to the site of a diseased tissue. In one exemplary embodiment, the targeting agent facilitates delivery of the vector composition to the site of a solid tumor. The type of targeting ligand is determined by the desired delivery site. The delivery site and the ability to couple the targeting ligand to the nanoparticle platform will dictate the type of targeting ligand that may be used and can be determined by one of ordinary skill in the art. In one exemplary embodiment, the targeting ligand is tumor necrosis factor (TNF). In certain embodiments, the presence of a targeting ligand is not needed to achieve site specific delivery.

The stealth agents of the present invention may comprise agents that protect the nanoparticle vector composition from absorption, digestion or other metabolic activity prior to the vector composition reaching its target. In certain exemplary embodiments, the stealth agent may help the vector compositions avoid detection and clearance by the reticuloendothelial system (RES). In certain embodiments, the stealth agent increases hydration of the vector composition. The stealth agent may also prevent the adsorption of certain blood components, such as opsonins, on the surface of the vector composition. In addition, the stealth agent may also sterically stabilize the vector composition against recognition and/or clearance by the RES. Exemplary stealth agents include, but are not limited to, polyethylene glycol (PEG), hydroxyethyl starch (HES/HAES), PolyPEG®, or rPEG any of which may be used in original form, thiolated or otherwise derivatized. Additional stealth agents include, thiolated polyoxypropylene polymers, thiolated block copolymers or triblock copolymers comprising polyoxyethylene/polyoxypropylene/polyoxyethylene blocks, as well as branched aminated PEGs, and methacrylamide polymers.

In one exemplary embodiment, the stealth agent is a branched aminated PEG. Branched aminated PEGS suitable for use in the present invention include but are not limited to two, three, four, five, six, seven, and eight branched aminated PEGs. In certain exemplary embodiments, the branched aminated PEG is a four arm branch aminated PEG. In another exemplary embodiment, the stealth agent is a methacrylamide polymer. In certain exemplary embodiments, the methacrylamide polymer is a N-(2-hyroxypropyl)methacrylamide (HPMA) polymer.

In certain exemplary embodiments, a separate stealth agent is not required. Instead, one or more active agents and any targeting ligands are assembled on a polymer backbone to form a functionalized polymer. One or more functionalized polymers may then be attached to a nanoparticle platform. In one exemplary embodiment, the polymer backbone comprises methacrylamide monomers. An exemplary methacrylamide polymer monomer includes, but is not limited to HPMA monomers. In one exemplary embodiment, the active agents and any targeting ligands are attached to a HPMA monomer to form functionalized monomers, wherein the functionalized monomers are then polymerized to form the functionalized polymers. In another exemplary embodiment, the functionalized polymer is a PolyPEG®. In certain exemplary embodiments the functionalized polymer may further comprise one or more stealth domains comprising unmodified monomers. In one exemplary embodiment, the stealth domain comprises unmodified HPMA monomers. In certain exemplary embodiments, the stealth domain is at the terminal end of the polymer opposite the site of attachment to the nanoparticle platform, and in certain other exemplary embodiments the stealth domain is contained within the functionalized polymer. In other exemplary embodiments, multiple stealth domains are placed at one or more locations throughout the functionalized polymer.

The functionalized polymers of the present invention may help the vector compositions avoid detection and clearance by the reticuloendothelial system (RES). In certain embodiments, the functionalized polymer increases hydration of the vector composition. The functionalized polymer may also prevent the adsorption of certain blood components, such as opsonins, on the surface of the vector composition. In addition, the functionalized polymer may also stericallly stabilize the vector composition against recognition and/or clearance by the RES. In certain exemplary embodiments, the vector composition may further comprise a stealth agent in addition to the functionalized polymer. In one exemplary embodiment, the stealth agent is an unmodified HPMA polymer.

The present invention comprises methods of active or targeted delivery of one or more active agents by administration of the nanoparticle vector compositions of the invention by known methods such as by injection or oral administration. In one embodiment, the present invention comprises methods for treating diseases, such as cancer or solid tumors, by administering the compositions of the present invention comprising agents that are known for the treatment of such diseases. Another embodiment comprises vector compositions comprising derivatized PEG, TNF (Tumor Necrosis Factor) and anti-cancer agents, associated with colloidal metal particles. In another embodiment, the present invention comprises methods for gene therapy by administering the compositions of the present invention comprising agents that are used for gene therapy, such as oligonucleotides, antisense oligonucleotides, vectors, ribozymes, siRNAs, DNA, mRNA, sense oligonucleotides, and nucleic acids.

### BRIEF DESCRIPTION OF THE DRAWINGS

FIG. 1A is a schematic of a mixing apparatus used to prepare a nanodrug in accordance with an example embodiment.

FIG. 1B is a schematic of a mixing apparatus used to prepare a stealth nanodrug in accordance with an example embodiment.

FIG. 2 is graph showing saturation binding of TNF to colloidal gold.

FIG. 3A is a graph showing the effect of TNF:gold binding ratios on the safety of cAu-TNF.

FIG. 3B is a graph showing the effects of TNF:gold binding ratios on the safety of cAu-TNF.

FIG. 3C is a chart showing the anti-tumor efficacy of cAu-TNF and native TNF.

FIG. 3D is a chart showing TNF distribution profiles after 1 hour.

FIG. 3E is a chart showing TNF distribution profiles after 8 hours.

FIG. 3F is a graph of pharmacokinetic profiles of native TNF and a cAu-TNF vector in MC38 tumor-burdened C57/BL6 mice.

FIG. 4, A-C, shows liver and spleen of mice treated with PT-cAu-TNF vectors (A), cAu-TNF vectors (B), and no treatment (C).

FIG. 5A is a graph showing gold distribution in various organs.

FIG. 5B is a graph showing TNF pharmacokinetic analysis.

FIG. 5C is a graph showing the intra-tumor TNF distribution over time.

FIG. 5D is a chart comparing the intra-tumor TNF concentrations with different formulation of the colloidal gold nanodrugs.

FIG. 5E is a graph showing the distribution of native TNF in various organs over time.

FIG. 5F is a graph showing the distribution of PEG-thiol stabilized cAU-TNF vector in various organs over time.

FIG. 6A is a graph comparing safety and efficacy of native TNF or PT-cAu-TNF vectors.

FIG. 6B is a graph comparing Native TNF and 20 K-PT-cAu-TNF safety and efficacy.

FIG. 6C is a graph comparing Native TNF and 30 K-PT-cAu-TNF safety and efficacy.

FIG. 7 is a schematic of a vector having multiple agents.

FIG. 8 is a schematic of an embodiment of a capture method for detecting a vector.

FIG. 9 is a graph showing TNF- and END-captured vectors exhibiting the presence of the second agent.

FIG. 10 is a graph showing TNF- and END-captured vectors exhibiting the presence of the second agent.

FIG. 11 is a schematic of a pegylating agent known as PolyPeg®.

FIG. 12 is a schematic of an exemplary 4-arm aminated PEG and exemplary 6-arm aminated PEG.

FIG. 13 is picture showing the saturation binding of colloidal gold with a 10 kD form a 4-arm branched PEG amine.

FIG. 14 is a picture demonstrating aminated PEG-induced stabilization of 27 nm colloidal gold nanoparticles.

FIG. 15 is a schematic showing a HPMA monomer and polymer comprising various HPMA monomers functionalized with 1) tyrosine, 2) a peptide, 3) a chelating agent for an imaging radionuclide, and 4) a chelating agent for a therapeutic radionuclide.

FIG. 16 is a schematic demonstrating the ability to increase the therapeutic payload of the vector compositions of the present invention by attaching multiple therapeutic agents to a functionalized polymer.

FIG. 17 is a schematic demonstrating the ability to increase the therapeutic payload of an example vector composition by attaching multiple therapeutic agents to a functionalized polymer.

FIG. 18 is a schematic showing an exemplary vector construct of the present invention wherein a targeting ligand, detection agents, and therapeutic agents are bound to an HPMA polymer, the HPMA polymer further comprising an unmodified segment for RES avoidance.

### DETAILED DESCRIPTION

The present invention comprises compositions and methods for the delivery of active agents. The present invention also comprises methods for making the compositions and administering the compositions in vitro and in vivo. In general, the present invention is directed to the assembly of one or more active agents, stealth agents, and optionally one or more targeting ligands on a nanoparticle platform to form nanotherapeutic vector compositions.

The delivery of agents is used for detection or treatment of specific cells or tissues. For example, the present invention may be used for imaging specific tissue, such as solid tumors. The delivery of agents may further be used for treatments of biological conditions, including, but not limited to, chronic and acute diseases, maintenance and control of the immune system and other biological systems, infectious diseases, vaccinations, hormonal maintenance and control, cancer, solid tumors and angiogenic states as well as other physiological disorders. Such delivery may be targeted to specific cells or cell types, or the delivery may be less specifically provided to the body, in methods that allow for low level release of the agent or agents in a nontoxic manner. In one exemplary embodiment, the nanotherapeutics vector compositions described herein comprises colloidal metal sol compositions. Descriptions and uses of metal sol compositions are taught in U.S. Pat. No. 6,274,552; and related patent applications, U.S. patent application Ser. Nos. 09/808,809; 09/935,062; 09/189,748; 09/189,657, and 09/803,123; and U.S. Provisional Patent Application 60/287,363, all of which are herein incorporated in their entireties. Also incorporated in their entirety within this application are U.S. Provisional Patent Applications 60/287,363, 60/974,310, 61/069,108, 61/123,796, 60/981,920, 61/040,022, 61/124,290, 61/126,899, 61/228,243, and 61/326,424.

In certain exemplary embodiments, the present invention is directed to nanoparticle vector compositions comprising nanoparticle platforms for use in novel diagnostics as well as methods for delivering therapeutic agents. In one exemplary embodiment, vector compositions of the present invention are used in methods of treatment or detection comprising accumulation of one or more active agents in a solid tumor. Though not wishing to be bound by any particular theory, it is thought that use of such compositions results in the vector composition trafficking to, and accumulating in, tumors.

Although the components of the nanoparticle vector compositions are described herein in terms of specific function and purposes, it is to be noted in certain exemplary embodiments, that the components may also serve more than one function. For example, as discussed further below, a gold nanoparticle may not only serve as a platform for assembling a vector composition, but it may also contribute a “stealth/protective function” by preventing metabolic degradation and/or clearance of the attached active agents. In certain exemplary embodiments, the unique chemistry that results from the presence of a gold particle, delays activation (or hydrolysis) of an agent or prodrug until the target site is reached. In certain other embodiments, the targeting ligand may contribute a therapeutic and/or diagnostic effect in addition to conferring site specificity to a vector composition. For example, in certain exemplary embodiments, TNF can direct a vector composition to specific in vivo sites, such as a site of solid tumor growth, while also conferring an additional therapeutic activity at the site of delivery.

A stealth or protective agent may be an agent that protects the nanoparticle vector composition from absorption, digestion or other metabolic activity prior to reaching its target. In one exemplary embodiments, the stealth agent comprises PEG or thiolated PEG. In another exemplary embodiment, the stealth agent comprises a branched aminated PEG. In yet another exemplary embodiment, the stealth agent comprises a methacrylamide polymer.

All methods of administration are contemplated by the present invention, including intravenous and oral administration. In one exemplary embodiment, when vector compositions of the present invention are administered intravenously or orally, the vector compositions are found in, or associated with, a tumor.

In one exemplary embodiment, the various components of the vector compositions are admixed, associated with or bound directly or indirectly to a nanoparticle platform. Admixing, associating and binding includes covalent and ionic bonds and other associations that allow for long term or short term association of the components with each other and with the nanoparticle platforms. Indirect binding includes binding through integrating molecules. The integrating molecules may be non-specific, such as a polylysine, which through its positive charge can associate with the surface of a nanoparticle platform and certain active agents, stealth agents, and targeting ligands. The integrating molecule may also be a binding pair, wherein one member of the binding pair is attached to the nanoparticle platform and the member of the binding pair is bound to the component to be attached to the nanoparticle platform. One exemplary binding pair is streptavidin/biotin. In one exemplary embodiment, the nanoparticle surface is bound with streptavidin and the surface of the component to be attached to the nanoparticle platform is bound with biotin. In another exemplary embodiment, the nanoparticle surface is bound with biotin the surface of the component to be attached to the nanoparticle platform is bound with streptavidin.

### Nanoparticle Platforms

The nanoparticle platforms of the present invention provide a base on which other molecules may be assembled to form the vector compositions of the present invention. The nanoparticle platforms may comprise materials such as, but not limited to, colloidal metals, gold nanoparticles, silver nanoparticles, silica nanoparticles, iron nanoparticles, metal hybrid nanoparticles such as gold/iron nanoparticles, nanoshells, gold nanoshells, silver nanoshells, gold nanorods, silver nanorods, metal hybrid nanorods, quantum dots, nanoclusters, liposomes, dendrimers, metal-lipsome particles, metal-dendrimer nanohybrids and carbon nanotubes. Attachment of the one or more active agents to a nanoparticle platform may slow the hydrolytic conversion of the active agents in the circulation. In addition, the nanoparticle platform may also facilitate the accumulation of the active agent at the site of disease (e.g. a solid tumor). In certain embodiments, the attachment of the active agents to the nanoparticle platform may also control the rate at which the agents are converted to their active from. In certain embodiments where the nanoparticle platform comprises a magnetic or paramagnetic material, the nanoparticle platform may facilitate detection of the vector compositions.

In one exemplary embodiment, the nanoparticle platform comprises a colloidal metal material. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the Al³⁺, Ru³⁺, Zn²⁺, Fe³⁺, Ni²⁺ and Ca²⁺ ions.

In one exemplary embodiment, the colloidal metal is gold. In certain embodiments, the colloidal gold is in the form of Au²⁺. In another exemplary embodiment, the colloidal gold is HAuCl₄. The colloidal gold particles may have a negative charge at an approximately neutral pH. It is thought that this negative charge prevents the attraction and attachment of other negatively charged molecules. In contrast, positively charged molecules are attracted to and bind to the colloidal gold particle. The colloidal gold may be employed in the form of a sol that contains gold particles having a range of particle sizes. In one exemplary embodiment, the size of the gold particles ranges from approximately 1 to approximately 100 nm; from approximately 1 to approximately 50 nm; from approximately 1 to approximately 40 nm; from approximately 1 to approximately 30 nm; from approximately 1 to approximately 20 nm; from approximately 1 to approximately 10 nm; from approximately 20 to approximately 30 nm; from approximately 20 to approximately 50 nm; from approximately 20 to approximately 70 nm; from approximately 30 to approximately 50 nm; from approximately 30 to approximately 70 nm; from approximately 30 to approximately 90 nm; from approximately 30 to approximately 100 nm; or from approximately 50 to approximately 100 nm.

In certain exemplary embodiments of the present invention, the gold nanoparticles may also function to control the rate at which a drug analog or prodrug is converted into its active form. Though not wishing to be bound by the following theory, it is thought that the gold in the nanoparticle contributes to a unique chemistry that prevents the conversion of such analogs to active drugs or agents in blood. Accordingly, the gold nanoparticles may contribute to the safety and efficacy of a drug by facilitating the use of lower doses of the drug. Gold nanoparticles may also contribute to the overall stability of a drug. Delayed hydrolysis, or delayed conversion of the inactive agent to the active agent is highly desirable as the possibility of indiscriminate action or decreased efficacy may be minimized. In one exemplary embodiment, the vector composition comprises colloidal gold, TNF, PEG and a prodrug. In one exemplary embodiment, the prodrug is a thiolated paclitaxel derivative. Suitable thiolated paclitaxel derivatives for use with the present invention are described in International Patent Application No. PCT/US08/82956 to Virginia Tech Intellectual Properties, which is incorporated herein by reference.

In another exemplary embodiment, the colloidal metal is colloidal silver in a sodium borate buffer, having a concentration of approximately 0.1% to approximately 0.001% of solution. In another exemplary embodiment, the colloidal silver is at a concentration of 0.01% solution in a sodium borate buffer.

Targeting Ligands/Molecules

In certain exemplary embodiments, targeting ligands may be used to facilitate site-specific delivery of the vector compositions. In certain other exemplary embodiments, the presence of a targeting ligand is not needed to achieve site-specific delivery. One or more targeting ligands may be directly or indirectly attached, bound or associated with the nanoparticle platform. These targeting ligands can be directed to specific organs, tissues, cells or cell types. Such targeting ligands may include any molecules that are capable of selectively binding to specific cells or cell types. In certain exemplary embodiments, the targeting ligand is one member of a binding pair and as such, selectively binds to its binding partner. Such selectivity may be achieved by binding to structures found naturally on cells, such as receptors found on the surface of the cellular membrane, nuclear membrane, or the membrane of an organelle. The binding pair member may also be introduced synthetically on the cell, cell type, tissue or organ. Targeting ligands may also include receptors or parts of receptors that bind to molecules such as antibodies, antibody fragments, enzymes, cofactors, substrates, and other binding pair members known to those skilled in the art. Targeting ligands may also be capable of binding to multiple types of binding partners. For example, the targeting ligand may bind to a class or family of receptors or other binding partners. The targeting ligand may also be an enzyme substrate or cofactor capable of binding several enzymes or types of enzymes.

In one exemplary embodiment, the targeting ligand is an antibody. Suitable targeting antibodies include monovalent and divalent single chain antibody fragments (scFv), as well as fusion protein constructs thereof. Pardridge (J Drug Targ 2010; 18 (3):157-167) provides an overview of IgG fusion proteins useful in targeted delivery to the brain. Olafsen and Wu provide an overview of techniques for generating and optimizing of antibody variants for cell specific targeting (Semin Nucl Med 2010; 40 (3):167-81).

In another exemplary embodiment, the targeting ligand is a targeting peptide. In yet another exemplary embodiment, the targeting peptide is a tumor specific targeting peptide such as, but not limited to, peptides containing RGD and/or NGR motifs, thrombospondin-1 (TSP-1) mimetic peptides, F3, integrin binding peptides, ED-B, and fibrin-fibronectin binding peptides. An overview of tumor targeting peptides is provided by Ruoslahti et al. (J Cell Biology 2010; 188 (6):759-68).

In another exemplary embodiment, the targeting ligand is a ligand for specific cell surface receptors. In many disease states, certain cell surface receptors are known to be up-regulated allowing for targeted delivery through ligands specific for those receptors. For example, ligands targeting human epidermal growth factor receptor-2 (HER-2) (Mamot et al. Cancer Research 2005; 65(24):11631-11638), folic acid receptor (Wang et al. International Journal of Pharmaceutics 2007; 337 (1-2):63-73), transferrin receptors (Choi et al. PNAS 2010; 107(3):1235-1240), and vasoactive intestinal peptide receptors (VIP-R) (Dagar et al. Journal of Controlled Release 2003; 91 (1-2):123-133) have been used for targeted delivery to cancer cells.

In another exemplary embodiment, the targeting ligand may be a nucleic acid-based targeting ligand. In certain exemplary embodiments, the targeting ligand may be an aptamer. Aptamers are nucleic acids sequences that adapt a specific secondary and tertiary structure and like antibodies exhibit specific molecular recognition. Aptamers specific to any number of target molecules can be designed using systematic evolution of ligand by exponential enrichment (SELEX) carried out in the presence of the target molecule. International Patent Application Publication no. WO/2009/014705 to Keefe et al. describes methods for the in vivo selection of aptamers that may be linked to therapeutic or diagnostic compositions. International Patent Application Publication No. WO/2009/090554 to Tavitian et al. describe a modified SELEX system for generating metastatic cell specific aptamer molecules. Farokhzad et al. provide an overview of the use of aptamers for cancer specific targeting (Expert Opin Drug Deliv 2006; 3 (3):311-24).

Specific examples of targeting ligands include, but are not limited to, Interleukin-1 (“IL-1”), Interleukin-1 beta (“IL-1beta”), Interleukin-2 (“IL-2”), Interleukin-3 (“IL-3”), Interleukin-4 (“IL-4”), Interleukin-5 (“IL-5”), Interleukin-6 (“IL-6”), Interleukin-7 (“IL-7”), Interleukin-8 (“IL-8”), Interleukin-9 (“IL-9”), Interleukin-10 (“IL-10”), Interleukin-11 (“IL-11”), Interleukin-12 (“IL-12”), Interleukin-13 (“IL-13”), Interleukin-14 (“IL-14”), Interleukin-15 (“IL-15”), Interleukin-16 (“IL-16”), Interleukin-17 (“IL-17”), Interleukin-18 (“IL-18”) Interleukin 21 (“IL-21”), B7, lipid A, phospholipase A2, endotoxins, staphylococcal enterotoxin B and other toxins, Type I Interferon, IFN gamma, Type II Interferon, Tumor Necrosis Factor (“TNF” or “TNFα”), Transforming Growth Factor-α (“TGF-α”), Lymphotoxin, Migration Inhibition Factor, Granulocyte-Macrophage Colony-Stimulating Factor (“CSF”), Monocyte-Macrophage CSF, Granulocyte CSF, vascular epithelial growth factor (“VEGF”), angiogenic factors, Angiogenin, transforming growth factor-β (“TGF-β”), carbohydrate moieties of blood groups, Rh factors, fibroblast growth factor and other inflammatory and immune regulatory proteins, hormones, such as growth hormone, insulin, glucagon, parathyroid hormone, luteinizing hormone, follicle stimulating hormone, and luteinizing hormone releasing hormone, endocrine hormones, cell surface receptors, antibodies, nucleic acids, nucleotides, DNA, RNA, sense nucleic acids, antisense nucleic acids, cancer cell specific antigens, MART, MAGE, BAGE, and molecular chaperones such as HSPs (Heat Shock Proteins), mutant p53; tyrosinase; antoimmune antigens; receptor proteins, glucose, glycogen, phospholipids, and monoclonal and/or polyclonal antibodies, basic fibroblast growth factor, enzymes, cofactors, enzyme substrates immunoregulatory molecules (i.e. CD40L), adhesion molecules (ICAM), vascular and neovascular markers (CD31 and CD34).

In one exemplary embodiment, the targeting ligand is TNF. The use of TNF as a targeting ligand may limit the biodistribution of the vector composition primarily to solid tumors and may contribute to the therapeutic effect enabling the simultaneous attack of not only the tumor cells present in a solid tumor, but also the host stromal cells that support and promote the tumor's growth. Accordingly, in certain embodiments including the embodiment wherein TNF is employed as the targeting agent, the targeting agent may also contributes to the therapeutic value of the vector composition in addition to its role as a targeting ligand.

### Stealth Agents

As used herein, “stealth agent” refers to any compound which when bound to the surface of the nanotherapeutic platform or molecule described herein assists in protecting the nanotherapeutic from being digested, absorbed, opsinized, or other metabolic activity prior to reaching its desired target. For example, thiolated polyethylene glycol hydrates the nanoparticle drug and in so doing, prevents its uptake and clearance by the reticuloendothelial system (RES).

In certain exemplary embodiments, the compositions of the present invention comprise as stealth agents, glycol compounds, preferably polyethylene glycol (PEG), (also known by those of ordinary skill in the art as polyoxyethylene or POE). The PEG may be a derivatized PEG. The present invention comprises compositions comprising derivatized PEG, wherein the PEG is 5,000 to 30,000 (daltons) MW. Derivatized PEG compounds are commercially available from sources such as SunBio, Seoul, South Korea. PEG compounds may be difunctional or monofunctional, such as methoxy-PEG (mPEG). Activated derivatives of linear and branched PEGs are available in a variety of molecular weights. As used herein, the term “derivatized PEG(s)” or “PEG derivative(s)” means any polyethylene glycol molecule that has been altered with either addition of functional groups, chemical entities, or addition of other PEG groups to provide branches from a linear molecule. Such derivatized PEGs can be used for conjugation with biologically active compounds, preparation of polymer grafts, or other functions provided by the derivatizing molecule.

In one exemplary embodiment, the PEG derivative is a polyethylene glycol molecule with primary amino groups at one or both of the termini. In another exemplary embodiment, the PEG derivative is methoxy PEG with an amino group on one terminus. Another type of PEG derivative which may be used with the present invention includes electrophilically activated PEGs. These PEGs are used for attachment of PEG or methoxy PEG (mPEG), to proteins, liposomes, soluble and insoluble polymers and a variety of molecules. Electrophilically active PEG derivatives include succinimide of PEG propionic acid, succinimide of PEG butanoate acid, multiple PEGs attached to hydroxysuccinimide or aldehydes, mPEG double esters (mPEG-CM-HBA-NHS), mPEG benzotriazole carbonate, and mPEG propionaldehyde, and niPEG acetaldehyde diethyl acetal.

In another exemplary embodiment, the derivatized PEG is a thiol derivatized PEG, or sulfhydryl-selective PEG. Branched, forked or linear PEGs can be used as the PEG backbone that has a molecular weight range of 5,000 to 40,000 daltons. In certain exemplary embodiments, the thiol derivatized PEG is derived from a PEG with maleimide functional group to which a thiol group can be conjugated. In one exemplary embodiment, the thiolated PEG derivative is methoxy-PEG-maleimide, with a molecular weight of 5,000 to 40,000 daltons.

Use of heterofunctional PEGs, as a derivatized PEG, is also contemplated by the present invention. Heterofunctional derivatives of PEG have the general structure X-PEG-Y. When the X and Y are functional groups that provide conjugation capabilities, many different entities can be bound on either or both termini of the PEG molecule. For example, vinylsulfone or maleimide can be X can be a vinylsulfone or maleimide functional group and Y can be an be a N-hyroxysuccinimide (NHS) functional group. For detection methods, X and/or Y can be fluorescent molecules, radioactive molecules, luminescent molecules or other detectable labels. Heterofunctional PEGs or monofunctional PEGs can be used to conjugate one member of a binding pair, such as PEG-biotin, PEG-Antibody, PEG-antigen, PEG-receptor, PEG-enzyme or PEG-enzyme substrate. PEG can also be conjugated to lipids such as PEG-phospholipids.

The stealth agents of the present invention may comprise other PEG like compounds including, but not limited to, thiolated polyoxypropylene polymers, thiolated block copolymers such as the PLURONICs, which are triblock copolymers comprising polyoxyethylene/polyoxypropylene/polyoxyethylene blocks. Examples of PLURONICS useful in the current invention include, but are not limited to, the following:

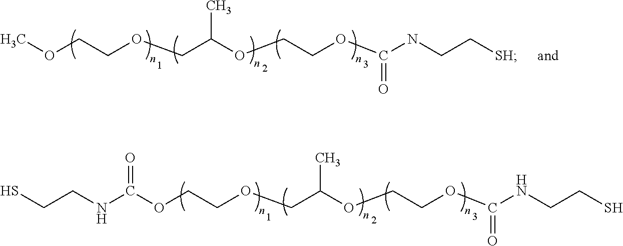

The molecular weight of the PLURONIC block polymer may be from, but not limited to, 1,000 to 100,000 daltons, more preferably between 2,000 and 40,000 daltons.

The polymer blocks are formed by condensation of ethylene oxide and propylene oxide, at elevated temperature and pressure, in the presence of a catalyst. There is some statistical variation in the number of monomer units, which combine to form a polymer chain in each copolymer. The molecular weights given are approximations of the average weight of copolymer molecules in each preparation and are dependent on the assay methodology and calibration standards used. It is to be understood that the blocks of propylene oxide and ethylene oxide do not have to be pure. Small amounts of other materials can be admixed so long as the overall physical chemical properties are not substantially changed. A more detailed discussion of the preparation of these products is found in U.S. Pat. No. 2,674,619, which is incorporated herein by reference in its entirety. (Also see, “A Review of Block Polymer Surfactants”, Schmolka I. R., J. Am. Oil Chemist Soc., 54:110-116 (1977) and Block and Graft Copolymerization, Volume 2, edited by R. J. Ceresa, John Wiley and Sons, New York, 1976

In one exemplary embodiment, the stealth agent comprises polyoxypropylene polymers (POP) that are functionalized, preferably with a thiol group. The preferred molecular weight of the PLURONIC block polymer is between 2,000 and 40,000 daltons. Also included in the present invention are branched polymers, including TETRONIC (PEO/PPO or PEO or PPO) copolymers), branched PEGs and various combinations of the disclosed block polymers. It is understood that linker molecules may be used between the nanoparticle platform surface and the polymer.

In another exemplary embodiment, the stealth agent comprises thiolated poly(vinylpyrrolidone) polymers (PVP) having the following general structure:

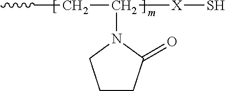

X indicates the site of an optional spacer arm that may be added to the polymer to provide better accessibility of the thiol group to the colloidal metal surface. The spacer arm may be comprised of, but is not limited to, the following propyl groups, amino acids, or polyamino acids. The preferred molecular weight of the PVP polymer is between approximately 1,000 and 100,000 daltons, more preferably between 5,000 and 40,000 daltons.

In yet another exemplary embodiment, the stealth agent is rPEG (Amunix, Mountain View Calif.). As used herein, rPEG generally refers to recombinant PEGylation technology generally involving the genetic fusing of a 300-600 amino acid unstructured protein tail to an existing pharmaceutical protein. Further description of rPEG may be found in United States Patent Publication No. 2008/0039341A1 which is herein incorporated by reference in its entirety.

In another exemplary embodiment, the stealth agent comprises a HES polymer, which is a hydroxyethyl starch (“HES”), a nonionic starch derivative, and is available by Fresenius Kabi, Inc. (Bad Homburg, Germany http://www.fresenius-kabi.com/). HES and HES derivatives may be derivatized and/or thiolated and bound to the colloidal gold nanoparticles.

In another exemplary embodiment, the stealth agent comprises branched aminated PEGs. Branched aminated PEGs suitable for use in the present invention may include, but are not limited to, two, three, four, five, six, seven, and eight branched aminated PEGS. In one exemplary embodiment, the vector compositions comprise colloidal metal sols associated with four arm aminated PEGS. In another exemplary embodiment, the vector compositions comprise colloidal metal sols associated with six arm aminated PEGS.

In certain other exemplary embodiments, a separate stealth agent is not required. Instead, one or more active agents and any targeting ligands are assembled on a polymer backbone to form a functionalized polymer. Multiple functionalized polymers may then be attached to a nanoparticle platform. In one exemplary embodiment, the polymer backbone comprises methacrylamide monomers. An exemplary methacrylamide polymer monomer includes, but is not limited to, N-(2-hydroxypropyl)methacrylamide (HPMA). Therapeutic agents, diagnostic agents, and targeting ligands can be attached to an HPMA monomer. The chemical make-up of the final functionalized polymer may then be customized by varying the molar ratio of the different monomer sub-types. Polymerization of the monomers may be achieved by a free radical reaction of the HPMA monomers in the presence of an initiator such as 2,2′-azobisisobuyronitrile. In order to facilitate attachment of active agents and targeting ligands to the functionalized polymer the HPMA monomers may be modified to contain free haloformyl, hydroxyl, carbonyl, aldehyde, carbonate, carboxylate, carboxylic acid, ether, ester, hydroperoxide, peroxide, amide, amine, imine, imide, azide, azo, cyanate, isocyanate, nitrate, nitrile, nitrite, nitro, nitroso, pyridine derivative, phosphine, phosphodiester, phosphonic acid, phosphate, sulfide, thiol, suflone, sulfonic acid, sulfoxide, thiocyanate, or disulfide groups. In one exemplary embodiment, an amino acid residue is attached to the HPMA monomer in order to facilitate attachment of active agents and targeting ligands. In another exemplary embodiment, active agents are attached to the HPMA monomers through a peptide linker. In another exemplary embodiment, the active agents are attached to the HPMA monomers through a hydrolytic linker. In one exemplary embodiment, the peptide linker is arginine-glycine-aspartate-cysteine-cysteine-cystein (RGD4C). Methods for polymerizing HPMA monomers containing different functional moieties are described in Mitra et al. (Nuclear Medicine and Biology 2006; 33:43-52), Mitra et al. (Pharm Res 2004; 21 (7):1153-9), Rathi et al. (J Polym Sci Part A: Polym Chem 1991; 29:1895-1902), and Nan et al. (J. Drug Targeting 2005; 13 (3):189-197).

In another exemplary embodiment, the functionalized polymer may be a PolyPEG® (Warwick Effect Polymers, Ltd., Coventry, United Kingdom). PolyPEG® is a novel pegylating agent for conjugation to therapeutic proteins, peptides and small molecules. PolyPEG®s comprise short PEG chains or “teeth” of varying molecular weight attached to a poly(methacrylate) backbone. The PEG teeth are attached to the poly(methacrylate) backbone via ester linkages, which can degrade over time. Several different chemistries can be used to covalently attached PolyPEG®s to therapeutic and diagnostic agents. These include a range of established chemistries for site-specific attachment to free cysteine residues, to the amines on lysine residues, or the terminal amine on biological molecules. Other conjugation chemistries may also be used. The structure of PolyPEG®s can be varied by; (1) the methacrylic backbone which determines the length of the comb; (2) the PEG chain length which determines the quantity of PEG on each tooth of the comb; and (3) the active end-group which determines the site of conjugation between the PolyPEG® and the target biomolecule.

The comb-like architecture of PolyPEG® provides an alternative approach to PEGylation by exploiting the properties of a structure that degrades to small units that are readily excreted over time. This allows their use at high total doses while avoiding potential toxicological problems associated with accumulation of larger molecular weight PEG chains in tissues. PolyPEG®s are similar to conventional PEGs in that they enhance the therapeutic effect of biological molecules by extending their circulatory presence. PolyPEG®s are capable of improving biological activity of certain peptides to a greater extent than convention PEGs. The PolyPEG® molecules can be tailored for a particular requirement for PEGylation of a range of therapeutic molecules.

The functionalized polymers of the present invention may help the vector compositions avoid detection and clearance by the reticuloendothelial system (RES). In certain embodiments, the functionalized polymer increases hydration of the vector composition. The functionalized polymer may also prevent the adsorption of certain blood components, such as opsonins, on the surface of the vector composition. In addition, the functionalized polymer may also sterically stabilize the vector composition against recognition and/or clearance by the RES. In certain exemplary embodiments, the vector composition may further comprise a stealth agent in addition to the functionalized polymer. In one exemplary embodiment, the stealth agent is an unmodified HPMA polymer. In certain exemplary embodiments, the functionalized polymer comprises a stealth domain comprising an unmodified polymer segment. In one exemplary embodiment, the stealth domain comprises an unmodified segment of an HPMA polymer. In another exemplary embodiment, the stealth domain is at the terminal end of the polymer opposite the polymer's site of attachment to the nanoparticle platform. In another exemplary embodiment, the stealth domain is found in one or more locations throughout the functionalized polymer. Functionalized polymers may be attached to the nanoparticle platform through functional groups such as free disulfide or thiol groups contained within a functionalized monomer on the terminal end of the functionalized polymer.

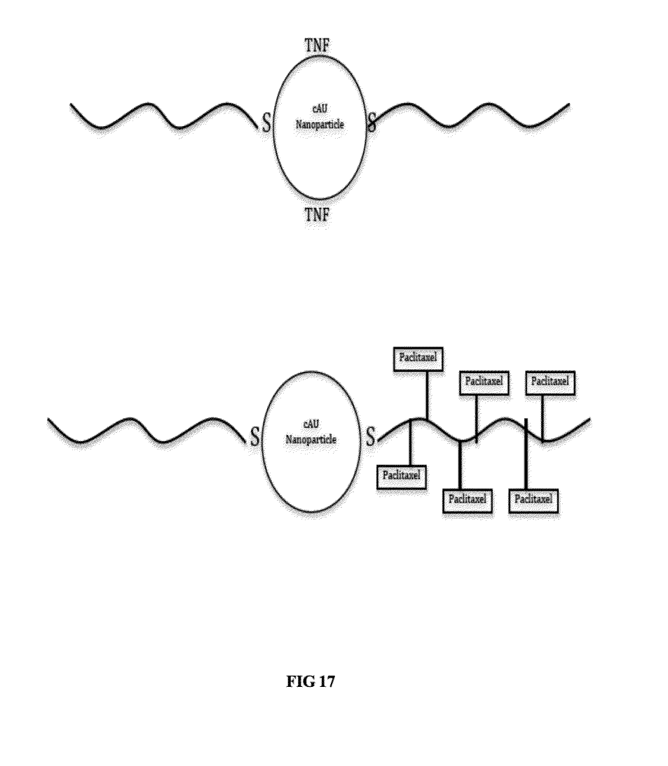

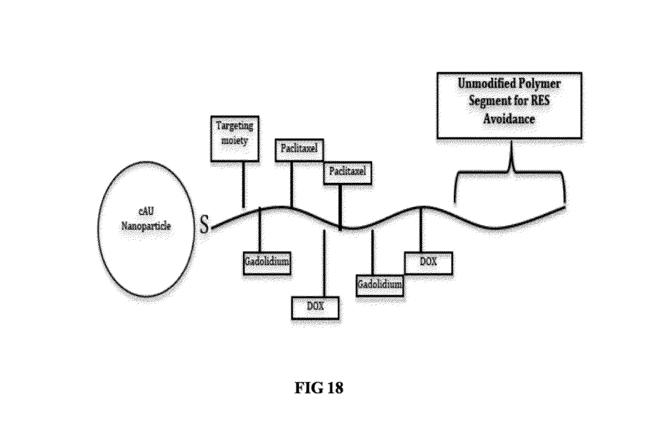

In certain exemplary embodiments, the use of functionalized polymers provides the ability to increase the therapeutic payload carried by each vector composition. As shown in FIG. 17, multiple therapeutic agents and/or diagnostic agents may be bound to a single functionalized polymer.

Any of the stealth agents discussed above maybe modified, derivatized (i.e. thiolated), aminated, or multi-aminated in connection with their use for the nanotherapeutic compositions of the present invention. In certain embodiments it may be preferred that the stealth agent comprises a polymer having a single terminal thiol group to facilitate its binding to a nanoparticle platform, such as colloidal gold.

### Active Agents

The active agents of the present invention can be any compound, chemical, therapeutic agent, pharmaceutical agent, drug, biological factors, fragments of biological molecules such as antibodies, proteins, lipids, nucleic acids or carbohydrates; nucleic acids, antibodies, proteins, lipids, nutrients, cofactors, nutriceuticals, anesthetic, detection agents or an agent that has an effect in the body. Such detection and therapeutic agents and their activities are known to those of ordinary skill in the art.

In one exemplary embodiment, the active agent comprises one or more detection agents such as dyes or radioactive materials that can be used for visualizing or detecting the sequestered vector compositions. Fluorescent, chemiluminescent, heat sensitive, opaque, beads, magnetic and paramagnetic materials are also contemplated for use as detectable agents that are associated or bound to nanoparticle platforms in the vector compositions of the present invention. In one exemplary embodiment, the detection agent is a MRI contrast agent such as gadolidium. In another exemplary embodiment, the contrast agent is a chelating agent for an imaging radionuclide, such as ^{99m}Tc. Examples of other suitable detection agents for use in the present invention can be found in the Molecular Imaging & Contrast Agent Database (MICAD) developed by the National Center for Biotechnology Information (NCBI). MICAD is a publicly available online resource of scientific information regarding molecular imaging and contrast agents, including those under development, in clinical trials, or commercially available for medical applications, that have in vivo data published in peer-reviewed journals. The database includes, but is not limited to, agents developed for positron emission tomography (PET), single photon emission computed tomography (SPECT), magnetic resonance imaging (MRI), ultrasound (US). computed tomography (CT), optical imaging, planar radiography, and planar gamma imaging. The database and a list of all detection and contrast agents within the database is publicly accessible at the NCBI website.

The following are non-limiting examples of some of the active agents that may be used in the present invention. In one exemplary embodiment, the active agent is a biological factor including, but not limited to, cytokines, growth factors, fragments of larger molecules that have activity, neurochemicals, and cellular communication molecules. Examples of such biological factors include, but are not limited to, Interleukin-1 (“IL-1”), Interleukin-1 beta (“IL-1beta”), Interleukin-2 (“IL-2”), Interleukin-3 (“IL-3”), Interleukin-4 (“IL-4”), Interleukin-5 (“IL-5”), Interleukin-6 (“IL-6”), Interleukin-7 (“IL-7”), Interleukin-8 (“IL-8”), Interleukin-10 (“IL-10”), Interleukin-11 (“IL-11”), Interleukin-12 (“IL-12”), Interleukin-13 (“IL-13”), Interleukin-15 (“IL-15”), Interleukin-16 (“IL-16”), Interleukin-17 (“IL-17”), Interleukin-18 (“IL-18”), Type I Interferon, Type II Interferon, Tumor Necrosis Factor (“TNFα”), Transforming Growth Factor-α (“TGF-α”), flT3, Lymphotoxin, Migration Inhibition Factor, Granulocyte-Macrophage Colony-Stimulating Factor (“CSF”), Monocyte-Macrophage CSF, Granulocyte CSF, vascular epithelial growth factor (“VEGF”), Angiogenin, transforming growth factor-β (“TGF-β”), fibroblast growth factor, angiostatin, endostatin, GABA, and acetyl choline.

Another type of active agent includes hormones. Examples of hormones include, but are not limited to, growth hormone, insulin, glucagon, parathyroid hormone, luteinizing hormone, follicle stimulating hormone, luteinizing hormone releasing hormone, estrogen, testosterone, dihydrotestoerone, estradiol, prosterol, progesterone, progestin, estrone, other sex hormones, and derivatives and analogs of hormones.

Yet another type of active agent includes pharmaceuticals. Any type of pharmaceutical agent can be employed in the present invention. For example, anti-inflammatory agents such as steroids and nonsteroidal anti-inflammatory agents, soluble receptors, antibodies, antibiotics, analgesics, angiogenic and anti-angiogenic agents, and COX-2 inhibitors, can be employed in the present invention. In one exemplary embodiment, the pharmaceutical is a chemotherapeutic agent. Non-limiting examples of suitable chemotherapeutic agents include taxol, paclitaxel, taxanes, vinblastin, vincristine, doxorubicin, acyclovir, cisplatin and tacrine and analogs thereof.

In another exemplary embodiment, the active agent is an immunotherapy agent. Non-limiting examples of immunotherapy agents, include inflammatory agents, biological factors, immune regulatory proteins, human and humanized antibodies, and immunotherapy drugs, such as AZT and other derivatized or modified nucleotides. Small molecules can also be employed as agents in the present invention.

Another type of agent includes nucleic acid-based materials. Examples of such materials include, but are not limited to, nucleic acids, nucleotides, DNA, RNA, tRNA, mRNA, sense nucleic acids, antisense nucleic acids, ribozymes, DNAzymes, protein/nucleic acid compositions, SNPs, oligonucleotides, vectors, viruses, plasmids, transposons, and other nucleic acid constructs known to those skilled in the art.

Other agents that can be employed in the invention include, but are not limited to, lipid A, phospholipase A2, endotoxins, staphylococcal enterotoxin B and other toxins, heat shock proteins, carbohydrate moieties of blood groups, Rh factors, cell surface receptors, antibodies, cancer cell specific antigens; such as MART, MAGE, BAGE, and HSPs (Heat Shock Proteins), radioactive metals or molecules, detection agents, enzymes and enzyme co-factors.

Other examples of active agents that may be used in the present invention are found in the following table. This table is not limiting in that other agents, such as the pharmaceutical equivalents of the following agents, are contemplated by the present invention.

TABLE I
Organisms and Selected Active Agents

| | |
|---|---|
| BACTERIA | |
| Mycobacterium tuberculosis | Isoniazid, rifampin, ethambutol, |
| | pyrazinamide, streptomycin, clofazimine, |
| | rifabutin, fluoroquinolones such as |
| | ofloxacin and sparfloxacin |
| Mycobacterium avium | Rifabutin, rifampin, azithromycin, |
| | clarithromycin, fluoroquinolones |
| Mycobacterium leprae | Dapsone |
| Chlamydia trachomatis | Tetracycline, doxycyline, erythromycin, |
| | ciprofloxacin |
| Chlamydia pneumoniae | Doxycycline, erythromycin |
| Listeria monocytogenes | Ampicillin |
| FUNGI | |
| Candida albicans | Amphotericin B, ketoconazole, fluconazole |
| Cryptococcus neoformans | Amphotericin B, ketoconazole, fluconazole |
| PROTOZOA | |
| Toxoplasma gondii | Pyrimethamine, sulfadiazine, clindamycin, |
| | azithromycin, clarithromycin, atovaquone |
| Candida albicans | Amphotericin B, ketoconazole, fluconazole |
| Cryptococcus neoformans | Amphotericin B, ketoconazole, fluconazole |
| VIRUS | |
| Herpes simplex virus type 1 | Acyclovir, trifluorouridine and other type 2 |
| | antiviral nucleoside analogs, foscornat, |
| | antisense oligonucleotides, and triplex- |
| | specific DNA sequences |
| Cytomegalovirus | Foscarnet, ganciclovir |
| HIV | AZT, DDI, DDC, foscarnat, viral protease |
| | inhibitors, peptides, antisense |
| | oligonucleotides, triplex and other nucleic |
| | acid sequences |
| Influenza virus types A & B | Ribavirin |
| Respiratory syncytial virus | Ribavirin |
| Varizella zoster virus | Acylcovir |

Additional therapeutic agents may include one or more of the following class of agents: antimetabolites of folic acid (such as but not limited to Aminopterin, Methotrexate, Pemetrexed, Raltitrexed), purine antimetabolites (such as but not limited to Cladribine, Clofarabine, Fludarabine, Mercaptopurine, Pentostatin, Thioguanine), pyrimidine antimetabolites (such as but not limited to Cytarabine, Decitabine, Fluorouracil/Capecitabine, Floxuridine, Gemcitabine, Enocitabine, Sapacitabine); alkylating Agents such as but not limited to nitrogen mustards (such as but not limited to Chlorambucil, Chlormethine, Cyclophosphamide, Ifosfamide, Melphalan, Bendamustine, Trofosfamide, Uramustine), nitrosoureas (such as but not limited to Carmustine, Fotemustine, Lomustine, Nimustine, Prednimustine, Ranimustine, Semustine, Streptozocin), platinum alkylating-like agents (such as but not limited to Carboplatin, Cisplatin, Nedaplatin, Oxaliplatin, Triplatin tetranitrate, Satraplatin), alkyl sulfonates such as but not limited to (Busulfan, Mannosulfan, Treosulfan), hydrazines (such as but not limited to Procarbazine; Triazenes such as but not limited to Dacarbazine, Temozolomide), Aziridines (such as but not limited to Carboquone, ThioTEPA, Triaziquone, Triethylenemelamine), spindle poisons/mitotic inhibitors (such as but not limited to the Taxanes (Docetaxel, Larotaxel, Ortataxel, Paclitaxel, Tesetaxel, Ixabepilone and epithilones, vinca alkaloids (such as but not limited to Vinblastine, Vincristine, Vinflunine, Vindesine, Vinorelbine), cytotoxic/antitumor antibiotics (such as but not limited to Anthracyclines (such as but not limited to Aclarubicin, Daunorubicin, Doxorubicin, Epirubicin, Idarubicin, Amrubicin, Pirarubicin, Valrubicin, Zorubicin), Anthracenediones (such as but not limited to Mitoxantrone, Pixantrone), Streptomyces based such as but not limited to (Actinomycin, Bleomycin, Mitomycin, Plicamycin) Hydroxyurea, Topoisomerase inhibitors (relating to Camptotheca such as but not limited to Camptothecin, Topotecan, Irinotecan, Rubitecan, Belotecan) Podophyllum (such as but not limited to Etoposide, Teniposide, others (such as but not limited to Altretamine, Amsacrine, Bexarotene, Estramustine, Irofulven, Trabectedin); cellular based therapies (such as but not limited to monoclonal antibodies against such as but not limited to Receptor tyrosine kinase Cetuximab, Panitumumab, Trastuzumab, CD20 such as but not limited to Rituximab, Tositumomab, Other such as but not limited to Alemtuzumab, Bevacizumab, Edrecolomab, Gemtuzumab, Infliximab, Basiliximab, Abciximab, Daclizumab, Gemtuzumab, Alemtuzumab, Rituximab, Palivizumab, Trastuzumab, Etanercept, Humanized antibodies and phage display antibodies, Fully human monoclonal antibodies against Cytokines and Cell surface and soluble receptors; Tyrosine kinase inhibitors such as but not limited to Axitinib, Bosutinib, Cediranib, Dasatinib, Erlotinib, Gefitinib, Imatinib, Lapatinib, Lestaurtinib, Nilotinib, Semaxanib, Sorafenib, Sunitinib, Vandetanib; Cyclin-dependent kinase inhibitors such as but not limited to Alvocidib, Seliciclib, Hormone based therapies such as but not limited dexamethasone, finasteride, tamoxifen, anti-androgen based hormone therapies, Delivery systems such as but not limited to, Viruses, Retroviruses, Adenoviruses, Adeno-associated viruses, Herpes viruses Pseudotyped viruses, Lentivirus Simian immunodeficiency virus coated with the envelope proteins, G-protein from Vesicular stomatitis virus, dendrimers designed to deliver genetic therapies (such as those designed to introduced the genes for cytokines such as but not limited to GMCSF, IL-2, TNF alpha, IL-12, IFN beta, chemosensitizing agents suicide genes such as but not limited to thymidine kinase, p53, sense such as RNA mRNA and antisense therapies including siRNAs; others therapeutics including but not limited to fusion protein (such as but not limited to Aflibercept, Denileukin diftitox), Anti-Inflammatory Therapies, Photosensitizers such as but not limited to, Aminolevulinic acid/Methyl aminolevulinate, Efaproxiral, Porphyrin derivatives (Porfimer sodium, Talaporfin, Temoporfin, Verteporfin), Retinoids (Alitretinoin, Tretinoin), Anagrelide, Arsenic trioxide, Asparaginase/Pegaspargase, Atrasentan, Bortezomib, Carmofur, Celecoxib, Demecolcine, Elesclomol, Elsamitrucin, Etoglucid, Lonidamine, Lucanthone, Masoprocol, Mitobronitol, Mitoguazone, Mitotane, Oblimersen, Omacetaxine, Sitimagene ceradenovec, Tegafur, Testolactone, Tiazofurine, Tipifarnib, and Vorinostat.

### Stabilization of Bound Prodrugs/Inactive Agents by the Nanoparticle Platform

Described in the current application is a concept wherein the mere binding of a putative pharmaceutical ingredient prodrug is protected from breakdown in the circulation. In turn the protected prodrug is conserved during its delivery to the site of disease (i.e., a solid tumor). Upon its arrival at the site of disease the prodrug is continually converted to active drug akin to a slow release depot.

The binding of a prodrug to a nanoparticle platform prevents the hydrolytic conversion of the prodrug to an active drug both in vitro and while in the circulation. In one exemplary embodiment, targeted delivery to a solid tumor is achieved via the attachment of a prodrug to a colloidal gold based vector composition of the present invention. Therefore, not only does attachment of the prodrug to a nanoparticle platform facilitate its delivery to the site of a solid tumor, but also facilitate sustained delivery of the active form of the drug over time.

### Binding and Delivery

General methods for binding agents to nanoparticle platforms may comprise the following steps. A solution of the active agent, functionalized polymer, stealth agent, and any targeting ligands is formed in a buffer or solvent, such as deionized water (diH₂O). The appropriate buffer or solvent will depend upon the agent to be bound. Determination of the appropriate buffer or solvent for a given agent is within the level of skill of the ordinary artisan. Determining the pH necessary to bind an optimum amount of agent to metal sol is known to those skilled in the art. The amount of agent bound can be determined by quantitative methods for determining proteins, therapeutic agents or detection agents, such as ELISA or spectrophotometric methods. The method of Horisberger (Biol Cellulaire 1979; 36:253-258), (1979), which is incorporated by reference herein, may be used to prepare vector compositions of the present invention. Another suitable method which may be used in preparation of the vector compositions of the present invention is disclosed in U.S. Patent Application Publication No. 2005/0175584 to Paciotti et al., which is also incorporated herein by reference.

The active agents, stealth agents, and any targeting ligands may be bound directly to the nanoparticle platform or indirectly via integrating molecules. An integrating molecule is a molecule that provides a site for the binding or association of two entities (e.g. a nanoparticle platform and an active agent). Integrating molecules that may be used in the present invention can either be specific or non-specific integrating molecules. The compositions of the present invention can comprise one or more integrating molecules. An example of a nonspecific integrating molecules is polycationic molecules such as polylysine or histones that are useful in binding nucleic acids. Polycationic molecules are known to those skilled in the art and include, but are not limited to, polylysine, protamine sulfate, histones or asialoglycoproteins. The present invention also contemplates the use of synthetic molecules that provide for binding one or more entities to the nanoparticle platforms. Specific integrating molecules comprise any members of binding pairs that can be used in the present invention. Such binding pairs are known to those skilled in the art and include, but are not limited to, antibody-antigen pairs, enzyme-substrate pairs, receptor-ligand pairs, and streptavidin-biotin. In addition to such known binding pairs, novel binding pairs may be specifically designed. A characteristic of binding pairs is the binding between the two members of the binding pair. Another desired characteristic of the binding partners is that one member of the pair is capable of binding or being bound to one or more of an agent or a targeting molecule, and the other member of the pair is capable of binding to the nanoparticle platform.

Where integrating molecules are employed in the present invention, the binding pH and saturation level of the integrating molecule is also considered in preparing the compositions. For example, where the integrating molecule is a member of a binding pair, such as streptavidin-biotin, the binding pH for streptavidin or biotin is determined and the concentration of the streptavidin or biotin bound can also determined.

In embodiments where an integrating molecule is employed, the integrating molecule is bound to, admixed or associated with the metal sol. The agent may be bound to, admixed or associated with the integrating molecule prior to the binding, admixing or associating of the integrating molecule with the metal, or may be bound, admixed or associated after the binding of the integrating molecule to the metal.

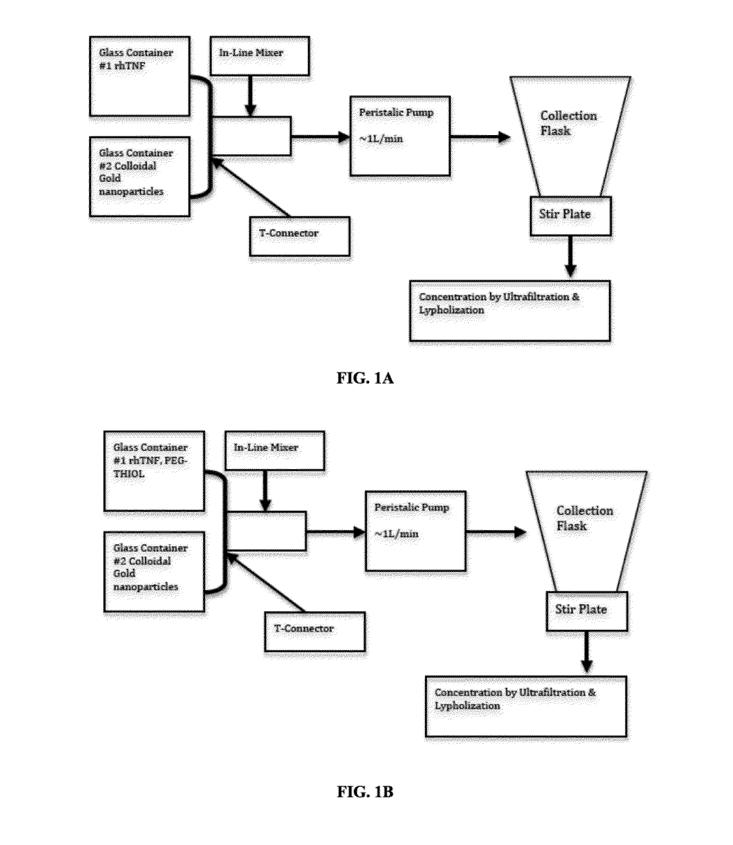

One method of binding an agent to a nanoparticle platform may comprise the following steps, though for clarity purposes only, the exemplary method disclosed refers to binding TNF, to colloidal gold. An apparatus was used that allows interaction between the particles in the colloidal gold sol and TNF in a protein solution. A schematic representation of the apparatus is shown in FIG. 1. This apparatus maximizes the interaction of unbound colloidal gold particles with the protein to be bound, TNF, by reducing the mixing chamber to a small volume. This apparatus enables the interaction of large volumes of gold sols with large volumes of TNF to occur in the small volume of a T connector. In contrast, adding a small volume of protein to a large volume of colloidal gold particles is not a preferred method to ensure uniform protein binding to the gold particles. Nor is the opposite method of adding small volumes of colloidal gold to a large volume of protein. Physically, the colloidal gold particles and the protein, TNF are forced into a T-connector by a single peristaltic pump that draws the colloidal gold particles and the TNF protein from two large reservoirs. To further ensure proper mixing, an in-line mixer is placed immediately down stream of the T-connector. The mixer vigorously mixes the colloidal gold particles with TNF, both of which are flowing through the connector at a preferable flow rate of approximately 1 L/min.

Prior to mixing with the agent, the pH of the gold sol is adjusted to pH 8-9 using 1 M NaOH. Highly purified, lyophilized recombinant human TNF is reconstituted and diluted in 3 mM Tris. Before adding either the sol or TNF to their respective reservoirs, the tubing connecting the containers to the T-connector is clamped shut. Equal volumes of colloidal gold sol and the TNF solution are added to the appropriate reservoirs. Preferred concentrations of agent in the solution range from approximately 0.01 to 15 μg/ml, and can be altered depending on the ratio of the agent to metal sol particles. Preferred concentrations of TNF in the solution range from 0.5 to 4 μg/ml and the most preferred concentration of TNF for the TNF-colloidal gold composition is 0.5 μg/ml.

Once the solutions are properly loaded into their respective reservoirs, the peristaltic pump is turned on, drawing the agent solution and the colloidal gold solution into the T-connector, through the in-line mixer, through the peristaltic pump and into a collection flask. The mixed solution is stirred in the collection flask for an additional hour of incubation.

In compositions comprising a stealth agent, whether derivatized or not, the methods for making such compositions comprise the following steps, though for clarity purposes only, the methods disclosed refer to adding PEG thiol to a metal sol composition. Any PEG, derivatized PEG composition or any sized PEG compositions or compositions comprising several different PEGs, can be made using the following steps. Following the 1-hour incubation taught above, a thiol derivatized polyethylene glycol (PEG) solution is added to the colloidal gold/TNF sol. The present invention contemplates use of any sized PEG with any derivative group, though preferred derivatized PEGs include mPEG-OPS S/5,000, thiol-PEG-thiol/3,400, mPEG-thiol 5000, and mPEG thiol 20,000 (Shearwater Polymers, Inc.). A preferred PEG is mPEG-thiol 5000 at a concentration of 150 μg/ml in water, pH 5-8. Thus, a 10% v/v of the PEG solution is added to the colloidal gold-TNF solution. The gold/TNF/PEG solution is incubated for an additional hour.

The colloidal gold/TNF/PEG solution is subsequently ultrafiltered through a 50K MWCO diafiltration cartridge. The 50K retentate and permeate are measured for TNF concentration by ELISA to determine the amount of TNF bound to the gold particles.

The compositions of the present invention can be administered to in vitro and in vivo systems. In vivo administration may include direct application to the target cells or such routes of administration, including but not limited to formulations suitable for oral, rectal, transdermal, ophthalmic, (including intravitreal or intracameral) nasal, topical (including buccal and sublingual), vaginal or parenteral (including subcutaneous, intramuscular, intravenous, intradermal, intratracheal, and epidural) administration. A preferred method comprises administering, via oral or injection routes, an effective amount of a composition comprising vectors of the present invention.

The formulations may conveniently be presented in unit dosage form and may be prepared by conventional pharmaceutical techniques. Pharmaceutical formulation compositions are made by bringing into association the metal sol vectors and the pharmaceutical carrier(s) or excipient(s). In general, the formulations are prepared by uniformly and intimately bringing into association the compositions with liquid carriers or finely divided solid carriers or both, and then, if necessary, shaping the product.

In certain exemplary embodiments, the present invention comprises methods of delivering an active agent to the site of a solid tumor, comprising administration of the vector compositions of the present invention. In one exemplary embodiment, the vector composition comprises a colloidal gold particle to which a stealth agent and one or more therapeutic and/or detection agents are attached. In one exemplary embodiment, the stealth agent is a branched aminated PEG. In one exemplary embodiment, the branch aminated PEG is a 4 branch aminated PEG. In another exemplary embodiment, the stealth agent is a HPMA polymer. In one exemplary embodiment, the therapeutic agent is paclitaxel. In another exemplary embodiment, the therapeutic agents are TNF and paclitaxel.

In another exemplary embodiment, the vector composition comprises a colloidal metal sols to which multiple functionalized polymers are attached, wherein the functionalized polymers contain multiple therapeutic and/or diagnostic agents attached to a polymer backbone. In one exemplary embodiment, collidal metal sol is colloidal gold, the functionalized polymer comprises a HPMA polymer backbone, and the therapeutic agent is paclitaxel. In another exemplary embodiment, the therapeutic agents are TNF and paclitaxel. In another exemplary embodiment, the functionalized polymer contains a stealth domain containing a section of unmodified HPMA monomers. In another exemplary embodiment, a one or more unmodified HPMA polymers are attached to the colloidal metal sol in addition to one or more functionalized polymers.

In one exemplary embodiment, the present invention comprises methods for the delivery of radioactive agents to the site of a solid tumor comprising administration of a vector composition. In one exemplary embodiment, the vector composition comprises a nanoparticle platform, radioactive or cytotoxic agents and stealth agents for delivery of radiation therapies to tumors. Historically, radioactive colloidal gold was used as a cancer therapy, principally for the treatment of liver cancer due to the anticipated uptake of colloidal gold by the liver cells. In one exemplary embodiment, the vector composition comprises a radioactive moiety coupled to a protein that is bound to colloidal metal, and further comprising a stealth agent. The radioactive vector composition of the present invention may be injected intravenously and traffics to the tumor and is not significantly taken up by the liver. In both compositions, it is believed that the ability of the stealth agent to concentrate the radioactive therapy in the tumor increases treatment efficacy while reducing treatment side effects. In one exemplary embodiment, the stealth agent is a branched aminated PEG. In another exemplary embodiment, the stealth agent is HPMA. In yet another exemplary embodiment, the radioactive moiety is attached to a HPMA polymer backbone to form a functionalized polymer, wherein the functionalized polymer is attached to the nanoparticle platform

In another exemplary embodiment, present invention further comprises methods for the detection of solid tumors comprising administering a vector composition of the present invention, wherein the vector composition comprises a detection agent. Detection agents include, but are not limited to, radioactive, radiation sensitive or reactive, such as light or heat reactive compounds, chemiluminescent or luminescent agents, fluororescent agents, magnetic agents, or other agents used for detection purposes. Methods of detection include, but are not limited to NMR, MRI, CAT or PET scans, visual examination, colorimetric, radiation detection methods, spectrophotometric, and protein, nucleic acid, polysaccharide or other biological agent detection methods.

In yet another exemplary embodiment the preset invention comprises methods for delivery of exogenous nucleic acids or genetic material into cells comprising administering vector compositions of the present invention, wherein the vector composition comprises one or more nucleic acid ligands. Nucleic acid ligands that may be delivered using the vector compositions of the present invention include may be DNA or RNA based and include, but are not limited to, oligonucleotides, aptamers, antisense oligonucleotides, small interfering RNA (siRNA), microRNA (miRNA), ribozymes and DNAzymes and expression vectors encoding a polypeptide. It is contemplated in the present invention that the nucleic acid ligands of the compositions may be internalized and used as detection agents or for genetic therapeutic effects, or the nucleic acids can be translated and expressed by the cell. The expression products can be any known to those skilled in the art and includes but is not limited to functioning proteins, production of cellular products, enzymatic activity, export of cellular products, production of cellular membrane components, or nuclear components. The methods of delivery to the targeted cells may be such methods as those used for in vitro techniques such as with cellular cultures, or those used for in vivo administration. In vivo administration may include direct application to the cells or such routes of administration as used for humans, animals or other organisms, preferably intravenous or oral administration. The present invention also contemplates cells that have been altered by the compositions of the present invention and the administration of such cells to other cells, tissues or organisms, in in vitro or in vivo methods.

In another exemplary embodiment, the present invention comprises methods for enhancing an immune response and increasing vaccine efficacy through the simultaneous or sequential targeting of specific immune cells comprising administering vector compositions of the present invention, wherein vector compositions comprise a targeting ligand that is a component-specific immunostimulating ligand. As used herein, component-specific immunostimulating ligand means an agent that is specific for a component of the immune system, such as a B or T cell, and that is capable of affecting that component, so that the component has an activity in the immune response. The component-specific immunostimulating ligand may be capable of affecting several different components of the immune system, and this capability may be employed in the methods and compositions of the present invention. The agent may be naturally occurring or can be generated or modified through molecular biological techniques or protein receptor manipulations. The compositions may also be used in methods for imaging or detecting immune cells. These methods comprise vector compositions that are capable of effecting the immune system, and include nanoparticle platforms associated with at least one of the following components, immune components specific targeting ligands, one or more agents, integrating molecules, and one or more types of stealth agents.

The activation of the component in the immune response may result in a stimulation or suppression of other components of the immune response, leading to an overall stimulation or suppression of the immune response. For ease of expression, stimulation of immune components is described herein, but it is understood that all responses of immune components are contemplated by the term stimulation, including but not limited to stimulation, suppression, rejection and feedback activities.

The immune component that is affected may have multiple activities, leading to both suppression and stimulation or initiation or suppression of feedback mechanisms. The present invention is not to be limited by the examples of immunological responses detailed herein, but contemplates component-specific effects in all aspects of the immune system.

The activation of each of the components of the immune system may be simultaneous, sequential, or any combination thereof. In one embodiment of a method of the present invention, multiple component-specific immunostimulating ligands are administered simultaneously. In this method, the immune system is simultaneously stimulated with multiple separate preparations, each containing a vector composition comprising a component-specific immunostimulating ligand. In one exemplary embodiment, the vector composition comprises the component-specific immunostimulating ligand associated with a nanoparticle platform. In another exemplary embodiment, the composition comprises the component-specific immunostimulating ligand associated with colloidal metal of one sized particle or of different sized particles and an antigen. Most preferably, the composition comprises the component-specific immunostimulating ligands associated with colloidal metal of one sized particle or of differently sized particles, antigen and PEG or PEG derivatives.

Component-specific immunostimulating ligands provide a specific stimulatory, up regulation, effect on individual immune components. For example, Interleukin-1α (IL-1α) specifically stimulates macrophages, while TNF-α (Tumor Necrosis Factor alpha) and Flt-3 ligand specifically stimulate dendritic cells. Heat killed Mycobacterium butyricum and Interleukin-6 (IL-6) are specific stimulators of B cells, and Interleukin-2 (IL-2) is a specific stimulator of T cells. Vector compositions comprising such component-specific immunostimulating ligands provide for specific activation of macrophages, dendritic cells, B cells and T cells, respectively. For example, macrophages are activated when a vector composition comprising the component-specific immunostimulating ligand IL-1α is administered. In one exemplary embodiment, IL-1α in association with colloidal metal. In another exemplary embodiment, IL-1α in association with colloidal metal and an antigen to provide a specific macrophage response to that antigen. Vector compositions can further comprise targeting molecules, integrating molecules, PEGs or derivatized PEGs.

Many elements of the immune response may be necessary for an effective immune response to an antigen. An embodiment of a method of simultaneous stimulation is to administer four separate preparations of compositions of component-specific immunostimulating agents comprising 1) IL-1α for macrophages, 2) TNF-alpha and Flt-3 ligand for dendritic cells, 3) IL-6 for B cells, and 4) IL-2 for T cells. Each component-specific immunostimulating agent vector composition may be administered by any routes known to those skilled in the art, and all may use the same route or different routes, depending on the immune response desired.

In another embodiment of the methods and compositions of the present invention, the individual immune components are activated sequentially. For example, this sequential activation can be divided into two phases, a primer phase and an immunization phase. The primer phase comprises stimulating APCs, preferably macrophages and dendritic cells, while the immunization phase comprises stimulating lymphocytes, preferably B cells and T cells. Within each of the two phases, activation of the individual immune components may be simultaneous or sequential. For sequential activation, an exemplary method of activation comprises administration of vector compositions that cause activation of macrophages followed by dendritic cells, followed by B cells, followed by T cells. An exemplary sequential activation method comprises the administration of vector compositions that cause simultaneous activation of the macrophages and dendritic cells, followed by the simultaneous activation of B cells and T cells. This is an example of methods and compositions of multiple component-specific immunostimulating ligands to initiate several pathways of the immune system.

The methods and compositions of the present invention can be used to enhance the effectiveness of any type of vaccine. The present methods enhance vaccine effectiveness by targeting specific immune components for activation. Vector compositions comprising at least component-specific immunostimulating ligand in association with a colloidal metal, one or more stealth agents and one or more antigens are used for increasing the contact between antigen and the specific immune component, such as macrophages, B or T cells. Examples of diseases for which vaccines are currently available include, but are not limited to, cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough, and yellow fever.

The combination of routes of administration and the vector compositions for delivering the antigen to the immune system is used to create the desired immune response. The present invention also comprises methods and compositions comprising various compositions of packaging systems, such as liposomes, microcapsules, or microspheres, that can provide long-term release of immune stimulating vector compositions. These packaging systems act as internal depots for holding antigen and slowly releasing antigen for immune system activation. For example, a liposome may be filled with a vector composition comprising the agents of an antigen and component-specific immunostimulating agent, bound to or associated with a colloidal metal. Additional combinations are colloidal gold particles studded with agents such as viral particles which are the active vaccine candidate or are packaged to contain DNA for a putative vaccine. The vector may also comprise one or more targeting molecules, such as a cytokine, integrating molecules and PEG derivatives, HES®, PolyPEG® or rPEG, and the vector is then used to target the virus to specific cells. Furthermore, one could use a fusion protein vaccine, which targets two or more potential vaccine candidates, and provide a vector composition vaccine that provides protection against two or more infectious microorganisms. The compositions may also include immunogens, which have been chemically modified by the addition of polyethylene glycol which may release the material slowly.

Use of such vaccination systems as described above are important in providing vaccines that can be administered in one dose. One dose administration is important in treating animal populations such as livestock or wild populations of animals. One dose administration is vital in treatment of populations for whom healthcare is rarely accessible such as the poor, homeless, rural residents or persons in developing countries that have inadequate health care. Many persons, in all countries, do not have access to preventive types of health care, such as vaccination. The reemergence of infectious diseases, such as tuberculosis, has increased the demand for vaccines that can be given once and still provide long-lasting, effective protection. The compositions and methods of the present invention provide such effective protection.

The methods and compositions of the present invention can also be used to treat diseases in which an immune response occurs, by stimulating or suppressing components that are a part of the immune response. Examples of such diseases include, but are not limited to, Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, Sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, and non-Hodgkin's lymphoma.

The present invention includes presentation of agents such as antigen and component-specific immunostimulating agents in a variety of different delivery platforms or carrier combinations. For example, a preferred embodiment includes administration of a vector composition comprising a metal colloid particle bound to agents such as an antigen and component-specific immunostimulating agents in a liposome or biodegradable polymer carrier. Additional combinations are colloidal gold particles associated with agents such as viral particles which are the vaccine antigen or which are viable viral particles containing nucleic acids that produce antigens for a vaccine. The vector compositions may also comprise targeting molecules such as a cytokine or a selected binding pair member which is used to target the virus to specific cells, and further comprises other elements taught herein such as integrating molecules or PEG or PEG derivatives. Such embodiments provide for a vaccine preparation that slowly releases antigen to the immune system for a prolonged response. This type of vaccine is especially beneficial for one-time administration of vaccines. All types of carriers, including but not limited to liposomes and microcapsules are contemplated in the present invention.

### Toxicity Reduction and Vaccine Administration

The vector compositions of the present invention may be used in methods for reducing the toxicity of certain toxic factors, such as certain therapeutics and antigens. The association of such toxic factors with the vector compositions of the present invention may be rendered less harmful or less toxic to non-toxic to the human or animal than when the agent is provided alone without the vector composition.

For example, it is known that Interleukin-2 (IL-2) displays significant therapeutic results in the treatment of renal cancer. However, the toxic side effects of administration of IL-2 result in the death of a significant number of the patients. In contrast, if a vector composition comprising at least IL-2 and a colloidal metal is administered, little or no toxicity is observed and a strong immune response occurs in the recipient. The doses previously used for IL-2 therapy have been on the order of 21×10⁶ units of IL-2 per 70 kg person per day (7×10⁶ units of IL-2 per 70 kg person TID). One unit equals approximately 50 picograms, 2 units equals approximately 0.1 nanograms, so 20×10⁶ units equals 1 milligram. In effect, the studies of the effects of the administration of agents described herein have included doses of more than 20 times higher than that previously given to humans. In another experiment, IL-2 (1 mg per 3 kg animal) was administered to 3 rabbits every third day for a two-week period, all the animals appeared to be clinically sick, and two of the animals died from the apparent toxic effects of the IL-2. When the same dose of IL-2 was used in vector compositions comprising colloidal gold and then administered to three rabbits for the same two-week period, no toxicity was observed and a significant antibody response resulted in all three animals. A “positive antibody response” as used herein is defined as a three to fourfold increase in specific antibody reactivity, as determined by direct ELISA, comparing the post-immunization bleed with the preimmunization bleed. A direct ELISA is done by binding IL-2 onto a microtiter plate, and determining the quantity of IgG bound to the IL-2 on the plate, by goat anti-rabbit IgG conjugated to alkaline phosphatase. Therefore, it is thought that the biological effects of the IL-2 remain. As the toxicity effects have been minimized, larger concentrations of IL-2 may be administered if necessary where a larger, more effective immune response is required.

The vector compositions may optionally include a pharmaceutically-acceptable carrier, such as an aqueous solution, or excipients, buffers, antigen stabilizers, or sterilized carriers. Also, oils, such as paraffin oil, may optionally be included in the composition. The vector compositions may further comprise a pharmaceutically acceptable adjuvant, including, but not limited to Freund's complete adjuvant, Freund's incomplete adjuvant, lipopolysaccharide, monophosphoryl lipid A, muramyl dipeptide, liposomes containing lipid A, alum, muramyl tripeptidephosphatidylethanoloamine, keyhole limpet hemocyanin. A preferred adjuvant for animals is Freund's incomplete adjuvant and Alum for humans, which preferably is diluted 1:1 with the compositions comprising a colloidal metal and an active agent.

In practicing these embodiments, the route by which the composition is administered is not considered critical. The routes that the composition may be administered according to this invention include known routes of administration, including, but are not limited to, subcutaneous, intramuscular, intraperitoneal, oral, and intravenous routes. In one exemplary embodiment, the route of administration is intravenous. In another exemplary embodiment, the route of administration is intramuscular. In yet another exemplary embodiment, the route of administration is oral.

When a vector composition comprising at least a colloidal metal and at least one agent was incubated with cells for 25 days, it was found that only 5% of the agent was released from the colloidal metal. Thus, it is theorized that circulation time alone does not explain the mechanism through which the agents are released from the complex in vivo. However, it has been found that the amount of agent released is, in part, related to the concentration of the complex in the body. When various dilutions of compositions were analyzed (CytELISA™ assay system CytImmune Sciences, Inc.), it was found that the more dilute solutions of the complex released a significantly greater amount of agent. For example, there was essentially no release of agent in a 1:100 dilution of the complex, whereas over 35,000 pg. of the agent was released in a 1:100,000 dilution of the composition.

Therefore, the lower the concentration of the composition in the larger solution, the greater the amount of agent released. The higher the concentration of the composition, the lower the amount of agent released. Thus, it is theorized that due to the continuous in vivo dilution of the compositions by blood and extracellular fluids, it is possible to achieve the same therapeutic effect by administering a lower dose of an agent to a patient than can be administered by previously known methods.

It is also theorized that the amount of agent released from the compositions of the present invention is related to the amount of agent initially bound to the nanoparticle platform. More agent is released in vivo from vector compositions having a greater amount of agents initially bound. Thus, the skilled artisan could control the amount of agents delivered by varying the amount of agent initially bound to the colloidal metal.

These combined properties provide methods by which a large amount of agents can be bound to a colloidal metal, thereby rendering the agent less toxic than if administered alone. Then, a small amount of the vector composition can be administered to a patient resulting in the slow release of the agent from the complex. These methods provide an extended, low dose of the agents for the treatment of diseases such as cancer and immune diseases.

The compositions of the present invention are useful for the treatment of a number of diseases including, but not limited to, cancer, both solid tumors as well as blood-borne cancers, such as leukemia; autoimmune diseases, such as rheumatoid arthritis; hormone deficiency diseases, such as osteoporosis; hormone abnormalities due to hypersecretion, such as acromegaly; infectious diseases, such as septic shock; genetic diseases, such as enzyme deficiency diseases (e.g., inability to metabolize phenylalanine resulting in phenylketanuria); and immune deficiency diseases, such as AIDS.

Methods of the present invention comprise administration of the vector compositions in addition to currently used therapeutic treatment regimens. Preferred methods comprise administering vector compositions concurrently with administration of therapeutic agents for treatment of chronic and acute diseases, and particularly cancer treatment. For example, a vector composition comprising the agent, TNF, is administered prior to, during or after chemotherapeutic treatments with known anti-cancer agents such as antiangiogenic proteins such as endostatin and angiostatin, thalidomide, taxol, melphalan, paclitaxel, taxanes, vinblastin, vincristine, doxorubicin, acyclovir, cisplatin and tacrine. All currently known cancer treatment methods are contemplated in the methods of the present invention and the vector compositions may be administered at different times in the treatment schedule as necessary for effective treatment of the cancer.

In one exemplary embodiment, the present invention comprises a method for treatment of drug-resistant tumors, cancer or neoplasms. These tumors are resistant to known anti-cancer drugs and therapeutics and even with increasing dosages of such agents, there is little or no effect on the size or growth of the tumor. Known in cancer treatment is the observation that exposure of such drug resistant tumor cells to TNF resensitizes these cells to the anti-cancer effect of these chemotherapeutics. Evidence has been published that indicates that TNF synergizes with topoisomerase II-targeted intercalative drugs such as doxorubicin to restore doxorubicin tumor cell death. Also interferon (IFN) is known to synergize with 5-fluorouracil to increase the chemotherapeutic activity of 5-fluorouracil. The present invention can be used to treat such drug-resistant tumors. A preferred method comprises administration of compositions comprising vectors having TNF and derivatized PEG bound to colloidal gold. With the pretreatment of a patient with a subclinical dose of TNF-cAu-PT, the tumor sequesters the TNF vector, sensitizing the cells to subsequent systemic chemotherapy. Such chemotherapies include, but are not limited to doxorubicin, other intercalative chemotherapies, taxol, 5-fluorouracil, mitaxantrone, VM-16, etoposide, VM-26, teniposide, and other non-intercalative chemotherapies. Alternatively, another preferred method comprises administration of vector compositions of the present invention comprising TNF and at least one other agent effective for the treatment of cancer. For example, a PT-cAU_{(TNF)}doxorubicin vector is administered to patients who have drug resistant tumors or cancer. The amount administered is dependent on the tumor or tumors to be treated and the condition of the patient. The vector composition allows for greater amounts of the chemotherapeutic agents to be administered and the vector also relieves the drug-resistant characteristic of the tumor.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### Preparation of Colloidal Gold Sols

Colloidal gold is produced by the reduction of chloroauric acid (Au⁺³; HAuCl₄), to neutral gold (Au⁰) by agents such as sodium citrate. The method described by Horisberger, (1979) was adapted to produce 34 nm colloidal gold particles. This method provided a simple and scalable procedure for the production of colloidal gold. Briefly, a 4% gold chloride solution (23.03% stock; dmc², South Plainfield, N.J.) and a 1% sodium citrate solution (wt/wt; J. T. Baker Company; Paris, Ky.) were made in de-ionized H₂O (DIH₂O). 3.75 ml of the gold chloride solution was added to 1.5 L of DIH₂O. The solution was vigorously stirred and brought to a rolling boil under reflux. The formation of 34 nm colloidal gold particles was initiated by the addition of 60 ml of sodium citrate. The solution was continuously boiled and stirred during the entire process of particle formation and growth as described below.

The addition of sodium citrate to the gold chloride initiated a series of reduction reactions characterized by changes in the color of the initial gold chloride solution. With the addition of the sodium citrate the color of the gold chloride solution changed from a golden yellow to an intermediate color of black/blue. The completion of the reaction was signaled by a final color change in the sol from blue/black to cherry red. After the final color change the solution was continuously stirred and boiled under reflux for an additional 45 minutes. Subsequently, the sol was cooled to room temperature and filtered through a 0.22 μm cellulose nitrate filter and stored at RT until use.

The formation of colloidal gold particles occurs in two stages: nucleation and particle growth. Particle nucleation was initiated by the reduction of Au⁺³ to Au⁰ by sodium citrate. This step is marked by a color change of the gold chloride solution from bright yellow to black. The continuous layering of free Au⁺³ onto the Au⁰ nuclei drives the second stage, particle growth. Particle size is inversely related to the amount of citrate added to the gold chloride solution: increasing the amount of sodium citrate to a fixed amount of gold chloride results in the formation of smaller particles, while reducing the amount of citrate added to the gold solution results in the formation of relatively larger particles.

Similar to the nucleation reaction, colloidal gold particle formation is also correlated with a change in the solution's color. However, unlike the initial reaction, this second color change is directly related to particle size. When small particles (i.e., 12-17 nm) are made the sol is orange to red in color; when medium sized particles (i.e., 20-40 nm) are made the sol appears red to burgundy in color and when large particles (i.e., 64-97 nm) are made the sols appear violet to brown in color. Critical to both particle nucleation and growth was the vigorous stirring of the reactants. Inadequate stirring at any step during the process resulted in the formation of heterogeneous particles with larger than predicted diameters.

TEM (transmission electron microscopy) and dual angle light scattering interrogation of the colloidal gold preparations revealed that the size of the particles in the colloidal gold preparations were very close to their theoretical size of 34 nm. The particles were homogenous in size with a mean particle diameter of 34-36 nm and a polydispersity measure averaging 0.11 (Table IV). In this state the colloidal gold particles stayed in suspension by their mutual electrostatic repulsion due to the negative charge present on each particle's surface. Exposing these naked particles to salt solutions (i.e., NaCl at a 1% v/v final concentration) caused them to aggregate and ultimately precipitate out of solution. This process was blocked or inhibited by binding proteins (e.g., TNF) or other agents to the particles' surface.

### Example 2

### Metal Sources

Experiments were performed to see if the source of the starting gold reactants for the formation of the colloid formation affected the colloidal gold compositions. Gold chloride was purchased from two different commercial sources: Degussa Metals Catalysts Cerdec (dmc²) and Sigma Chemical Company. Both gold preparations were analyzed for the presence of contaminating metals as well as other substances. The results from these studies are listed in Table II. Although the gold concentrations in each preparation were within reported values, it is clear that the Sigma preparation contains higher levels of Mg, Ca and Fe.

TABLE II
Purity of gold chloride salts used to generate colloidal gold

| Element | dmc² | Sigma |
|---|---|---|
| Na | <25 ppm | >21 ppm |
| Mg | <25 ppm | >60 ppm |
| Ca | <25 ppm | >60 ppm |
| Fe | <25 ppm | >60 ppm |

TEM of the particles revealed further differences between the particles made with different gold chloride sources, from Sigma and from dmc². The colloidal gold sols were manufactured as described above and observed used TEM. After cooling, 10 ml of the sol was centrifuged to concentrate the particles. The resultant supernatant was removed by aspiration and the colloidal gold pellet was re-suspended by gentle tituration. The pellet was prepared for transmission electron microscopy following standard procedures.

The particles made with the Sigma gold chloride are translucent with apparent striations. The striations have been reported to be due to the presence of trace contaminants, such as those identified above. In contrast, the particles made with dmc² gold chloride are electron dense with very few striations.

### Example 3

### Generation of Colloidal Gold Sols Using Sigma and dmc² Gold Chloride

The above data suggested that the gold chloride from dmc² contained lower levels of contaminating elements. To determine the effect of these two qualitatively different sources of gold chloride, colloidal gold sols were generated using the two different sources of the salt. The procedure for creating the colloidal gold particles follows the procedure originally described by Horisberger, and in Example 1. Briefly, a 4% gold chloride (in water) solution was made from the dmc² and Sigma stock preparations. 3.75 ml of each solution was added to individual flasks each containing 1.5 L of water. The solution was brought to a rolling boil, kept boiling under reflux, and vigorously stirred. 22.5 ml of a 1% sodium citrate solution was added to each flask. The solutions in both flasks were kept boiling until the well-described process of colloidal gold formation was complete, signaled by a color conversion from gold to black to cherry red. Once the sols turned a cherry red, they were allowed to boil under reflux, with constant stirring, for an additional 45 min. After cooling, the sols were filtered through a 0.22 mm nitrocellulose filter and stored at room temperature until use.

A qualitative comparison of the two sols was made with a standard laboratory spectrophotometer, running a UV/VIS wavelength scan. The results revealed that the two batches of sols contained colloidal gold particles with a similar mean diameter, as indicated by the wavelength where the sol exhibits the greatest absorbance. However the most striking difference between the two preparations is that the sol made with the dmc² material had 3-times the number of particles as those made with the Sigma material. In addition, it appeared that the distribution around the lambda max is wider in the Sigma preparation than for the dmc² preparation, indicating that the particles generated with the Sigma salt are more heterogeneous than those generated with the dmc² gold chloride.

### Example 4

### Analytica Comparison of the Colloidal Gold Sols

The above qualitative differences were confirmed by quantitative particle characterization with a Brookhaven Particle Sizer. For these studies the samples of particles from each gold source were prepared according to manufacturer's instructions. The data are presented below in Tables II and III. The data confirmed that the particles in both preparations are of approximately the same size (34-37 nm). Nevertheless the sols made with the dmc² material have a 3-fold higher particle density than those made with the Sigma material. In addition, the particles made with the Sigma gold chloride preparation are 2.5 times more heterogeneous (i.e., have a larger value for their polydispersity) than the particles made with the dmc² material (Table IV).

TABLE III
Variable wavelength analysis of colloidal gold
sols generated with dmc² and Sigma gold Chloride

| Sample | 1 Max | Absorbance | 1 @ ½ Max |
|---|---|---|---|
| dmc² | 526 nm | 2.8899 | 576 |
| Sigma | 529 nm | 1.0513 | 587 |

TABLE IV
Mean particle size and distribution of colloidal
gold sols generated with dmc² and Sigma gold chloride

| | Particle Size | Mean Polydispersity |
|---|---|---|
| dmc² | 34.0 | 0.096 |
| Sigma | 36.9 | 0.230 |

### Example 5

### Determination of the pH Binding Optimum

The binding of proteins to colloidal gold is known to be dependent on the pH of the colloid gold and protein solutions. The pH binding optimum of TNF to colloidal gold sols was empirically determined. This pH optimum was defined as the pH that allowed TNF to bind to the colloidal gold particle, but blocked salt-induced (by NaCl) precipitation of the particles. Naked colloidal gold particles are kept in suspension by their mutual electrostatic repulsion generated by a net negative charge on their surface. The cations present in a salt solution cause the negatively charged colloidal gold particles, which normally repel each other, to draw together. This aggregation/precipitation is marked by a visual change in the color of the colloidal gold solution from red to purple (as the particles draw together) and ultimately black, when the particles form large aggregates that ultimately fall out of solution. The binding of proteins or other stabilizing agents to the particles' surface will block this salt-induced precipitation of the colloidal gold particles.

The pH optimum of TNF binding to colloidal gold was determined using 2 ml aliquots of 34 nm colloidal gold sol whose pH was adjusted from pH 5 to 11 (determined by using pH strips) with 1N NaOH. TNF (Knoll Pharmaceuticals; purified to homogeneity) was reconstituted in diH₂O to a concentration of 1 mg/ml and further diluted to 100 μg/ml in 3 mM TRIS base. To determine the pH binding optimum for TNF, 100 μl of the 100 μg/ml TNF stock was added to the various aliquots of pH-adjusted colloidal gold. The TNF was incubated with the colloid for 15 minutes. Subsequently 100 μl of a 10% NaCl solution was added to each of the aliquots to induce particle precipitation. The optimal binding pH was defined as the pH, which allowed TNF to bind to the colloidal gold particles, while preventing the particles' precipitation by salt.

### Example 6

### Saturation Binding Studies

Based on the data obtained from the pH binding study, the pH of 34 nm colloidal gold sol was adjusted to pH 8 with 1 N NaOH. The sol was divided into 1 ml aliquots to which increasing amounts (0.5 to 4 μg of TNF) of a 100 μg of TNF/ml solution were added. After binding for 15 minutes the samples were centrifuged at 7,500 rpm for 15 minutes. A 10 μl sample of the supernatant was added to 990 μl of EIA assay diluent (provided as part of a commercial EIA kit for TNF measurement; CytImmune Sciences, Inc., Rockville, Md.). The remainder of the supernatant was removed by aspiration and the colloidal gold pellet was resuspended to its original volume in a PEG 1450/diH₂O solution pH 8. 10 μl of the resuspended pellet was added to 990 μl of EIA assay diluent. The reconstituted pellet and supernatant solutions were serially diluted and analyzed for TNF concentration by a quantitative commercial EIA (CytImmune Sciences, Inc, Rockville, Md.).

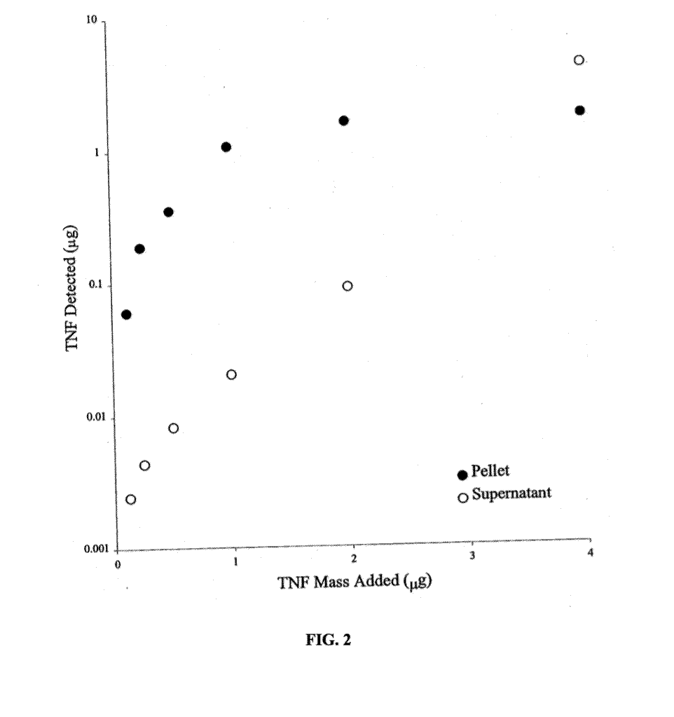

Using the data from the pH binding study, the pH of 50 ml of colloidal gold was adjusted between 8.0-9.0. At this pH the binding of TNF to a fixed volume of colloidal gold exhibited saturation kinetics (FIG. 2). As shown in FIG. 2, at 0.5 μg of TNF/ml of gold sol virtually all the TNF was bound to the colloidal gold particles with an insignificant amount (2-5%) present as free TNF in the supernatant. This colloidal gold-TNF complex precipitated in the presence of salt, indicating that this concentration of TNF did not fully coat the colloidal gold particles and is a sub-saturating dose of TNF. By increasing the TNF concentration the amount of TNF bound to the colloidal gold particles progressively increased with relatively little change in the amount of free TNF measured in the supernatant. This increase in particle-bound TNF paralleled the increase in the particles' stability against salt-induced precipitation. Saturation of the colloidal gold particles with TNF occurred when all the binding sites on the surface of the particles were bound with TNF. Saturation of the colloidal gold particles occurred at a binding concentration of 4 μg/ml (FIG. 2). Binding at doses above 4 μg/ml resulted in increasing amounts of free TNF measured in the supernatant.

In FIG. 2, saturation binding of TNF to colloidal gold is shown. 50 ml of 34 nm colloidal gold sol was pH adjusted to 8 using 1 NaOH, and then divided into 1 ml aliquots. Increasing volumes of a stock TNF (100 μg/ml in 3 mM TRIS) solution were added to the aliquots and allowed to bind for 15 minutes. The samples were centrifuged at 7500 rpms for 15 minutes. A 10 ml sample of the supernatant was diluted in a Tris buffered saline milk solution (assay diluent). The remainder of the supernatant was removed by aspiration and the colloidal gold pellet was resuspended by gentle tituration. 10 μl of the resuspended pellet was diluted in assay diluent. Both pellet and supernatant samples were serially diluted and measured for TNF concentration by EIA (CytImmune Sciences, Inc.).

### Example 7

### Large Scale Production of the Various Colloidal Gold Vectors

The in vivo assessment of the colloidal gold-TNF particles, also referred to as vectors, required the scaling-up of all manufacturing procedures. Large (8 L) batches of colloidal gold were manufactured as described above. The manufacturing procedure of the colloidal gold sols was adapted to 8 L colloidal gold production. A reflux apparatus (Kontes Glass, Vineland, N.J.) was used to generate 8 L of colloidal gold for in vivo experiments. Briefly, 8 L of diH₂O was heated to a rolling boil. 20 ml of gold chloride was added through one port, followed by the addition of 320 ml of sodium citrate. The color change of the resultant sol was the same as that seen in the smaller preparation. Once the cherry red color was achieved the sol was allowed to cool overnight, and was then sterile filtered as described above.

The particles made using large scale amounts were essentially the same as particles made using bench scale methods. See Table V.

TABLE V
Characterization of bench scale and large scale
preparations of 34 nm colloidal gold sols by dynamic light scattering.

| Preparation | Measured Size nm | Polydispersity |
|---|---|---|
| Bench Scale/1.5 L | 36 | 0.131 |
| Large Scale/(8.0 L) | 34 | 0.096 |

Next, the uniform coating of the colloid particles had to be accomplished. This was an important consideration since analysis demonstrated that the binding between the colloidal gold particles and the TNF molecules was nearly instantaneous. Consequently, simply adding a concentrated protein solution to a large volume of gold would result in particles that were differentially coated with TNF. To optimize the interaction of the particles and TNF molecules, apparatus that allowed complete interaction between the colloidal gold sol and the TNF solution was used. A schematic representation of the apparatus is shown in FIG. 1. The apparatus reduced the mixing volume between the naked colloidal gold particles and TNF by drawing each component into a small mixing chamber (a T-connector). The colloidal gold particles and the TNF solutions were physically drawn into the T-connector by a single peristaltic pump that drew the colloidal gold particles and the TNF protein from two large reservoirs. To further ensure proper mixing, an in-line mixer (Cole-Palmer Instrument Co., Vernon Hills, Ill.) was placed immediately downstream of the T-connector. The mixer vigorously mixed the colloidal gold particles with TNF, both of which were flowing through the connector at a flow rate of approximately 1 L/min.

Prior to mixing, the pH of the gold sol was adjusted to pH 8 using 1 N NaOH, while the recombinant human TNF was reconstituted and prepared in 3 mM Tris. The solutions were added to their respective sterile reservoirs using a sterile closed tubing system. Equal volumes of the colloidal gold sol and the TNF solution were added to the appropriate reservoirs. Since the gold and TNF solutions were mixed in equal volumes, the initial starting TNF concentration for each test vector was double the final concentration. For example, to make 4 L of a 0.5 μg/ml solution of cAu-TNF, 2 L of colloidal gold were placed in the gold reservoir, while 2 L of a 1 μg/ml TNF solution was added to the TNF container.

Once the solutions were properly loaded into their reservoirs, the peristaltic pump was activated, drawing the TNF and the colloidal gold solutions into the T-connector, through the in-line mixer, the peristaltic pump, and into a large collection flask. The resultant mixture was stirred in the collection flask for 15 minutes. After this binding step, 1 ml samples from each of the formulations were collected and tested for salt precipitation. A 1.0 μg/ml and a 4.0 μg/ml preparations were processed as described below, while a third solution, a second 0.5 μg/ml preparation, was treated by adding mPEG-thiol 5,000 (10% v/v addition of a 150 mg/ml stock in diH₂O) at a final concentration of 15 mg/ml. This third solution, a PEG-thiol-colloidal gold-TNF (PT-cAu-TNF) solution, was incubated for an additional 15 minutes. Two other PT-cAu-TNF formulations were made using a 20,000 and a 30,000 MW form of PEG-Thiol. During these studies additional controls were tested for comparison including PEG-Thiol/naked colloidal gold or the 4 mg/ml cAu-TNF vector.

Colloidal gold bound TNF in each preparation was separated from free TNF by diafiltration through a 50,000 MWCO BIOMAX diafiltration cartridge (Millipore Corporation, Chicago, Ill.). An aliquot of the permeate (i.e., free TNF) was removed and set aside for TNF determination. For mass balance determination, the total volume of the permeate was measured. The retentate, which contained the TNF bound colloidal gold, was sterile filtered through a 0.22 micron filter and a 10 ml aliquot was taken for TNF analysis. The remainder of the retentate was frozen at −80° C. for storage. Subsequent to the determination of the TNF concentrations, a solution of native TNF was manufactured in 3 mM Tris and used as the control for the in vivo studies.

### Example 8

### Initial Formulation of the Colloidal Gold TNF Vector

This series of experiments was designed to determine the effect of various TNF:colloidal gold binding ratios on the in vivo biologic activity of the colloidal gold TNF vector. Three different formulations of the colloidal gold TNF vector were made based on the data generated from the TNF-binding-to-colloidal-gold saturation curve. The three vectors were generated by binding TNF at 1, 2 or 4 μg of TNF/ml of colloidal gold solution. These three vectors differed in their ability to remain colloidal following the addition of salt. The 1 μg/ml vector precipitated immediately (i.e., the color of the colloidal changed from cherry red to black) upon the addition of the salt solution. In contrast the color of the 2 μg/ml vector turned from red to purple, indicating an aggregation of the colloidal gold particles. Finally, the 4 μg/ml preparation remained red after the addition of salt, indicating that the particles remained colloidal and did not interact. Although the colloidal nature of the particles in the 1 and 2 μg/ml vectors was altered by their exposure to salt, they remained stable when incubated with normal human plasma. These data suggested that plasma factors, most likely blood borne proteins, bound to the particle and immediately stabilized it against precipitation. Thus exposing these vectors to blood prevented their precipitation and allowed for their investigation in vivo.

Comparative safety studies of the three cAu-TNF vectors and native TNF were done in MC-38 tumor-burdened C57/BL6 mice. The toxicity profile of native TNF was dose-dependent. 5 μg of native TNF/mouse caused piloerection and diarrhea within 1-2 hours of injection. With increasing doses of native TNF more severe toxicities were observed. At a dose of 15 micrograms of TNF/mouse, 50% of the animals became hypothermic and unresponsive, and ultimately died within 24 hours. The mice were scored at different times after injection using the following toxicity rating scale: 0=normal activity; 1=piloerection; 2=loose stools; 3=lethargy; 4=unresponsive; and 5=death.

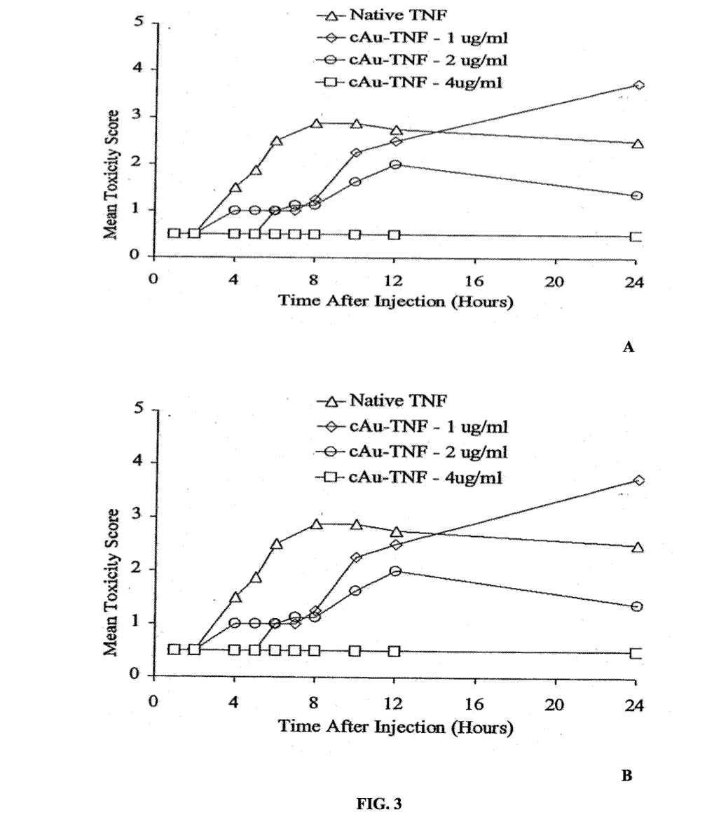

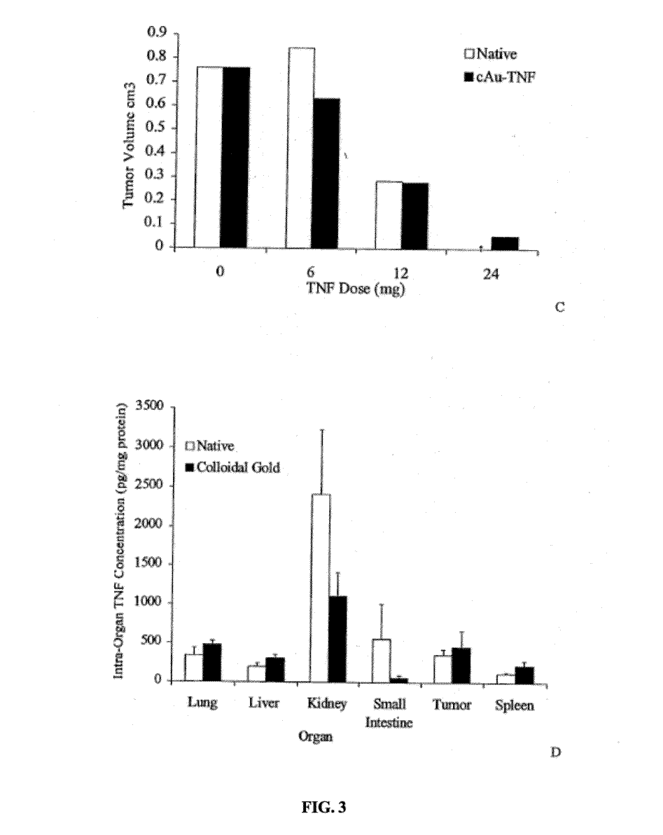

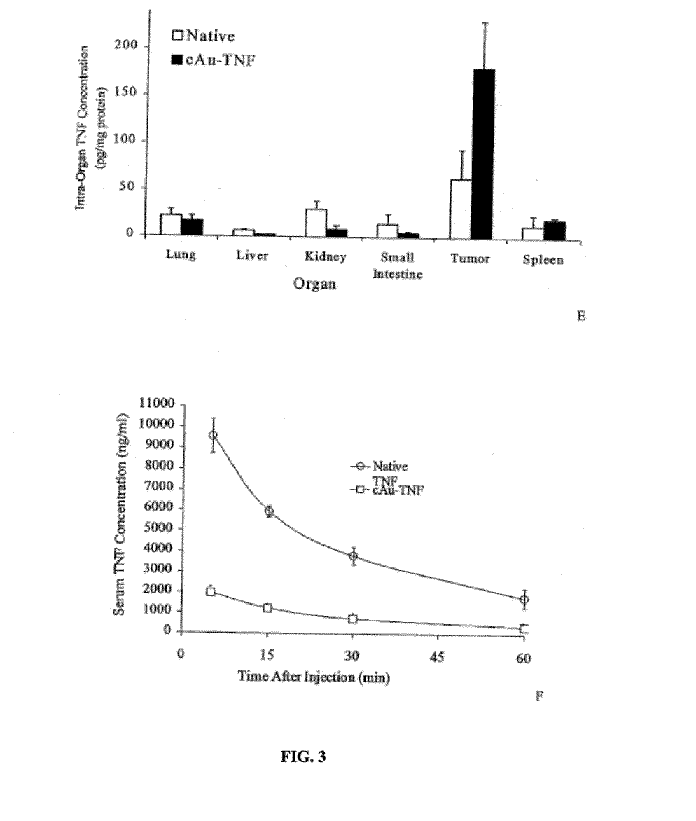

Although these three cAu-TNF vectors were biologically similar to native TNF preparations in the in vitro bioassay, their toxicity profiles were quite different in the C57/BL6-MC-38 tumor model. Increasing the initial binding concentration of TNF from 1.0 to 4.0 μg/ml increased the relative safety of the cAu-TNF vector (FIG. 3A). Mice injected with 15 μg of native TNF had a 50% mortality rate. A 15 μg injection of the 1.0 μg/ml cAu-TNF vector also caused a 50% mortality rate. In contrast, mice receiving 15 μg of the cAu-TNF bound at 2.0 μg/ml had a reduced mortality rate of 25%. Finally, none of the mice injected with 15 μg of the 4.0 μg/ml cAu-TNF preparation died. This last group of animals exhibited only transient toxicities that resolved within 8 hours of treatment.

FIG. 3A shows the effect of TNF:gold binding ratios on the safety of the cAu-TNF vector. Three different colloidal gold TNF vectors were generated based on their relative degree of TNF saturation of the colloidal gold particles. MC-38 tumor burdened C57/BL6 mice (n=4/group) were intravenously injected with 15 μg of either native TNF, not bound to a gold vector, or one of the three cAu-TNF vectors. The mice were scored at various points after the injection using the toxicity rating scale described. The percent survival for the treatments were: Native TNF=50%, 1 μg/ml cAu-TNF vector=25%, 2 μg/ml cAu-TNF vector=75% and 4 μg/ml cAu-TNF vector=100%.

A second dose escalation and safety study with the 4.0 μg/ml cAu-TNF vector indicated that this composition was safer on a dose-to-dose basis when compared to native TNF (FIG. 3B). Treatment with 12 micrograms or 24 micrograms of this cAu-TNF vector per mouse resulted in significant tumor reduction (FIG. 3C). In effect, this cAu-TNF vector increased the relative safety of any given dose of TNF and at a maximally tolerated dose improved the treatment's efficacy. These safety and efficacy data suggested that this cAu-TNF vector effectively increased the therapeutic index for TNF, since the drug's efficacy was maintained while its safety was improved.

FIG. 3B shows the dose escalation and toxicity of native TNF and 4 μg/ml cAu-TNF in MC-38 tumor-burdened C57/BL6 mice. MC-38-tumored C57/BL6 mice (n=4/group/dose) were intravenously injected with increasing doses of native TNF or the 4 μg/ml cAu-TNF vector. Mice were scored using the toxicity rating scale described. The percent of the animals surviving native TNF treatment at 6, 12, and 24 μg/mouse were 100%, 75% and 0% respectively. All animals receiving the cAu-TNF treatment survived. * p≦0.05.

FIG. 3C shows a comparison of the anti-tumor efficacy of native TNF and the 4 μg/ml cAu-TNF vector in MC-38 tumor-burdened C57/BL6 mice. The anti-tumor responses for the various treatment groups described in FIG. 3B were measured by determining three dimensional (L×W×H) tumor measurements 10 days after treatment. Data are presented as the mean±SEM of tumor volume (in cm³) for the various groups. All animals receiving the 24 μg native TNF treatment died within 24 hours of treatment. *p≦0.05 versus untreated controls.

These data strongly suggested a preferred composition for the cAu-TNF vector, which was then tested for its biodistribution. Over time, the biodistribution of TNF was different between those animals treated with native TNF and those treated with cAu-TNF. One hour after injection, mice receiving native TNF had higher levels of TNF in the kidney compared to cAu-TNF treated mice (FIG. 3D). In contrast, eight hours after injection, mice receiving the colloidal gold formulation had higher levels of TNF in the tumor (FIG. 3E). Thus it seemed that the cAu-TNF vector was improving safety and maintaining efficacy by targeting the delivery of TNF to the tumor.

FIGS. 3D and 3E show the comparison of the TNF distribution profiles in MC-38 tumor burdened C57/BL6 mice intravenously injected with 15 μg native TNF or the 4 μg/ml cAu-TNF vector. MC-38 tumor-burdened C57/BL6 mice (n=4/group/treatment/time point) were intravenously injected with 15 μg of native TNF or the 4 μg/ml cAu-TNF vector. A group of animals were sacrificed either 1 (FIG. 3D) or 8 hours (FIG. 3E) after injection and organs were collected. The organs were flash frozen at −80° C. and stored until analyzed. The organs were quickly defrosted by addition 1 ml of PBS (containing 1 mg/ml of bacitracin and PMSF) and homogenized using a polytron tissue disrupter. The homogenate was centrifuged at 5000 rpms and the resultant supernatant was analyzed for TNF concentration and total protein as described above. Data are presented as the mean±SEM from four organs per time point.

Autopsy of the animals revealed a potential problem with this cAu-TNF vector. The dramatic black color of the liver and the spleen of cAu-TNF vector treated mice argued that part of the improved safety may have been due to the vector's uptake and clearance by these organs. Further studies revealed that this uptake was rapid, often occurring within 5 minutes after intravenous injection. Visual inspection of these organs suggested that the black color of these organs was not different from the black precipitates formed when naked colloidal gold particles were exposed to salt. Also, it is unlikely that the black color of these organs was due to trapped blood in these organs since the animals were heparinized and extensively perfused prior to organ collection. These data suggested that a majority of the vector was rapidly cleared by the components of the RES, leading to the conclusion that the cAu-TNF vector was not optimized.

Additional evidence supporting this hypothesis was derived from pharmacokinetic studies that compared TNF levels between native TNF and two cAu-TNF vectors. Contrary to expectations, administration of the 4 μg/ml cAu-TNF vector resulted in lower initial serum levels than native TNF (FIG. 3F). The TNF levels of those mice given the cAu-TNF vector were consistently 2-5 fold lower than that measured in animals receiving native TNF. This difference in PK was more pronounced with a 0.5 μg/ml cAu-TNF vector, where the serum levels of TNF were nearly 10-fold lower than that seen with the native preparation. Mice receiving cAu-TNF vectors, regardless of the formulation (i.e., 0.5 or 4.0 μg/ml), consistently exhibited lower blood levels of TNF than mice receiving the native protein. Taken together, the vector biodistribution and PK data argued that uptake by the RES needed to be significantly reduced or eliminated for this vector to effectively target the tumor.

FIG. 3F shows a comparison of the pharmacokinetic profiles of native TNF or the 4 μg/ml cAu-TNF vector in MC38-tumor burdened C57/BL6 mice. MC-38 tumor burdened mice (n=3/group/time point) were intravenously injected with 10 μg of either native TNF or the 4 μg/ml cAu-TNF vector. At the indicated time points the mice were anesthetized and bled through the retro-orbital sinus. The blood samples were allowed to clot and were centrifuged at 14,000 rmps. The resultant sera samples were analyzed for TNF concentration using a commercial EIA for TNF. Data are presented as the mean±SEM serum concentration from three mice per time point. *p<0.05.

### Example 9

### Vectors to Avoid Clearance of the RES and Target the Delivery of TNF to Solid Tumors

Recognition and clearance of foreign objects by the RES has been seen with other drug carriers. For liposomes and biodegradable polymers this problem was addressed by surface modifications using a variety of PEG stabilizers as well as block co-polymers, such as polaxamer and polaxamine. Numerous stabilizers, including those used in liposome formulations (e.g., carbowax 20M, tetronic 407, pluronic 908) were added to the 4.0 μg/ml cAu-TNF vector. None of the reagents effectively blocked the vector's uptake by the RES.

Next, the amount of TNF bound per particle was reduced. TNF was first bound to the colloidal gold particles at a sub-saturating dose (i.e., 0.5 μg/ml). Thiol-derivatized polyethylene glycol (PEG-Thiol; MW=5,000) was then added to the particles. This small, linear PEG-Thiol reagent was chosen because thiol groups could bind directly to the particle's surface, presumably in between the molecules of TNF. This new vector was tested in the MC-38 tumor burdened C57/BL6 mice.

This composition of the colloidal gold bound TNF vector was formulated by binding TNF and an additional agent to the same particle of colloidal gold. This vector was formed by first binding TNF to colloidal gold at a subsaturating dose of 0.5 μg/ml. A derivatized PEG was then added to the vector. The derivatized PEG was a thiol-derivatized polyethlylene glycol (methoxy PEG-Thiol, MW: 5000 daltons, PEG-Thiol, Shearwater Corp., Huntsville, Ala.). The final concentration of PEG-Thiol was 15 μg/ml, which was added as a 10× concentrate in diH₂O. Thiol-derivatized PEGs are good components for colloidal gold vectors since the thiol group binds directly to the surface of the colloidal gold particles. A 5,000 daltons thiol-PEG was the first thiol-derivatized PEG to be tested. Additionally, efficacy experiments using mPEG-thiol with MWs of 20,000 and 30,000 daltons were performed as described below.

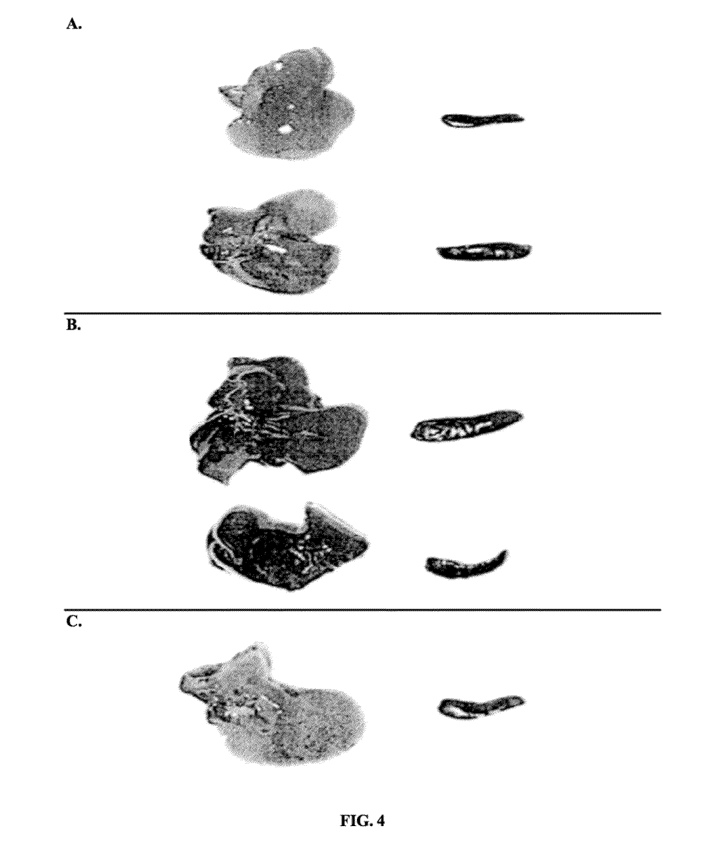

The biodistribution profile seen following the administration of the PEG-Thiol modified cAu-TNF (PT-cAu-TNF) vector was different from those observed with the previous cAu-TNF vectors. With this new vector, the liver and spleen did not visibly take up the PT-cAu-TNF vector, (FIG. 4A) as occurred with the 0.5 and 4.0 μg/ml cAu-TNF vectors (FIG. 4B). FIG. 4C shows an untreated liver and spleen. As striking as the inhibition of the RES uptake, was the apparent accumulation of the PT-cAu-TNF vector in the MC-38 tumor, since the tumors acquired the bright red/purple color of the colloidal gold particle within 30-60 minutes of vector's administration. The sequestration continued throughout the time course of the study and was coincident with the accumulation of TNF in the tumor and extended blood residence time of TNF.

Unlike the black color of the gold that accumulated in the liver and spleen following the 0.5 μg/ml and 4.0 μg/ml cAu-TNF vector treatment, the color of the gold observed accumulating in the tumor following the administration of the PT-cAu-TNF was reddish-purple. This difference is significant because it indicates that the gold particles remained in a colloidal state during their residence in the circulation and their accumulation in the tumor. Interestingly, the pattern of PT-cAu-TNF accumulation in and around the tumor site changed with time. The PT-cAu-TNF was initially (i.e., 0-2 hours) sequestered solely in the tumor. With time, vector staining was apparent on the skin and on the surrounding ventral tissues of the mouse. During the blunt dissection of sacrificed animal's tumor, it was observed that the extra-tumor staining in these animals was restricted to the dermal layer where the tumor cells were initially implanted. Minimal staining was present on the underlying muscle bed on which the tumor rested. This observation suggested that the peripheral staining may be due to the accumulation of the vector in the blood vessels, possibly new blood vessels, feeding the tumor mass. Currently, it is unknown whether the staining represents an active sequestration of the drug in these blood vessels or the passive accumulation due to tumor saturation with the vector.

To determine whether the staining reflected a hemorrhagic response cause by TNF, the staining pattern mice receiving a 15 micrograms injection of the 4 μg/ml cAu-TNF vector or native TNF was compared with those receiving the same dose of the PT-cAu-TNF vector. Mice treated with the 4 μg/ml cAu-TNF vector began to exhibit the tumor scar formation which typically follows intravenous administration of TNF. A similar pattern of scarring was observed following native TNF treatment. The pattern of the scar staining observed with the native TNF or the 4 μg/ml cAu-TNF vector treatments was clearly distinct from the pattern of staining observed following PT-cAu-TNF vector treatment. Further evidence that the staining pattern observed following the administration of the PT-cAu-TNF vector was obtained from mice receiving PEG-Thiol colloidal gold particles initially bound with murine serum albumin (MSA). The PT-cAu-MSA vector caused staining of the tumor like that of the PT-cAu-TNF vector albeit at a much slower rate. The staining of the tumor was similar in color to that observed with PT-cAu-TNF treatment. However the change in tumor coloration was only evident after 4 hours of treatment, compared with the 30-60 minute color change observed with the PT-cAu-TNF vector. Furthermore, the intensity of the staining was lower than that observed with the PT-cAu-TNF vector.

FIG. 4 shows inhibition of the RES-mediated uptake of the colloidal gold TNF vector by PEG-Thiol vectors. The PT-cAu-TNF vector was developed using specified ratios of TNF and PEG-Thiol as described. After binding, the vector was concentrated by diafiltration and analyzed for TNF concentration by EIA. 15 micrograms of the PT-cAu-TNF vector was intravenously injected into MC-38 tumor-burdened C57/BL6 mice. The mice were sacrificed 5 hours after the injection and perfused with heparinized saline. The livers (on left of picture) and spleens were photographed.

### Example 10

### Pharmacokinetic and Distribution Analysis

In general, these experiments comprise native TNF, cAu-TNF (4 mg/ml), or PT-cAu-TNF, (0.5 mg/ml) vectors that were generated as described above. Depending on the study, 5-20 micrograms of native or one of the cAu-TNF vectors were intravenously injected, through the tail vein, of MC-38 tumor-burdened mice. Mice were bled at 5, 180, and 360 minutes after injection through the retro-orbital sinus. The blood was allowed to clot and the resultant serum was collected and frozen at −20° C. for batch TNF analysis by EIA (CytImmune Sciences, Inc.). At selected time points, various organs were collected and flash frozen. To determine TNF content, the organs were defrosted, homogenized, and centrifuged at 14,000 rpms for 15 minutes. The supernatant was analyzed for TNF concentration as described above, as well as for total protein by determining the sample's absorbance a 280 nm. Organ TNF concentrations were normalized to total protein.

### A. Gold Distribution

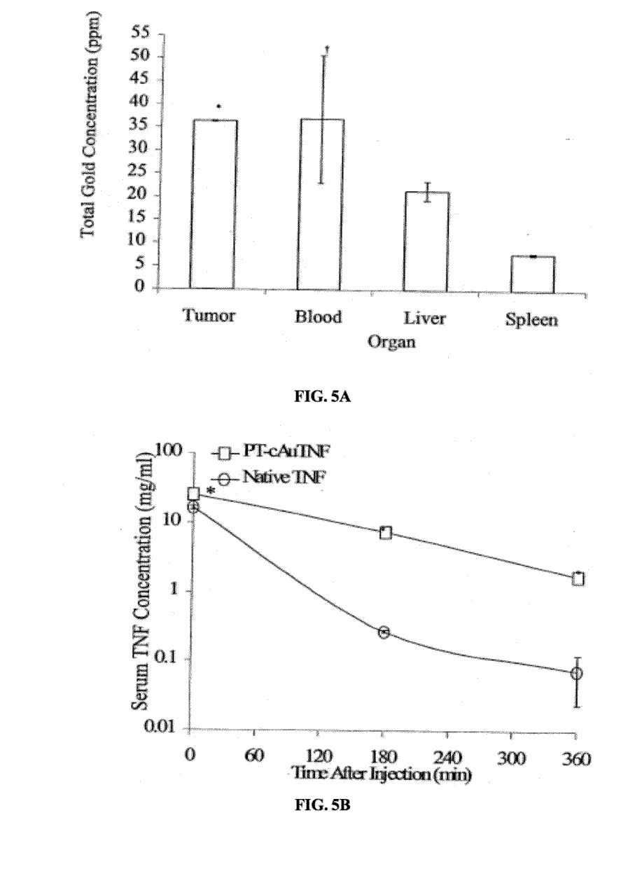

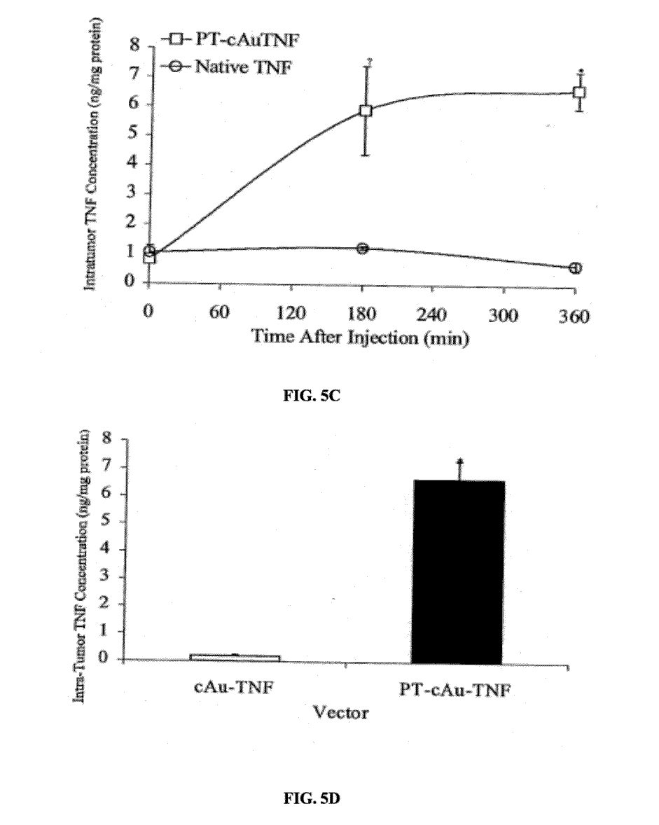

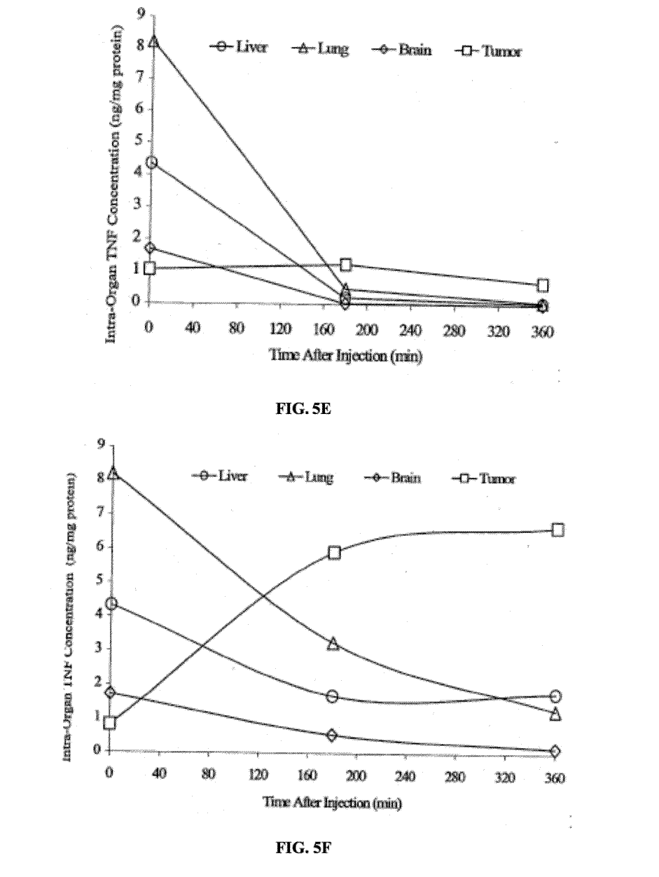

Various organs, including liver, lung, spleen, brain, and blood, were examined for the presence of elemental gold following the intravenous injection of 15 micrograms of the PEG-Thiol stabilized 0.5 μg/ml cAu-TNF vector. The mice were sacrifice 6 hours the injection; blood was collected and the various organs harvested, including liver, spleen, and tumor. After removal, the organs were digested in aqua-regia (3 parts concentrated HCl to 1 part concentrated nitric acid) to extract the gold present in these organs. The extraction was carried out over 24 hours, after which, the samples were centrifuged at 3500 rpms for 30 minutes. The supernatants were analyzed for the presence of total organ gold concentration by inductively coupled plasma spectroscopy. The results are reported as total organ gold concentration (in ppm) in FIG. 5A. The results demonstrate that the intra-tumor concentration of gold was nearly 2-fold higher than that measured in liver and nearly 7-fold higher than that found in the spleen. Although this pattern suggests that the vector was retained in the tumor compared to other organs, we observed that the highest level of gold was still in the circulation of these animals.

In FIG. 5A, gold distribution in various organs of MC-38 tumor burdened C57/BL6 mice is shown. The supernatants were analyzed for the presence of total organ gold concentration by inductively coupled plasma spectroscopy. The results are reported as total organ gold concentration (in ppm) for 3 mice per organ. *p<0.05 versus liver and spleen; † p<0.05 versus spleen.

### B. Distribution and Pharmacokinetics of TNF

The sequestration of colloidal gold within the tumor mass was paralleled by the prolonged presence of the drug vector in the circulation as well as the active accumulation of TNF in the tumor. Unlike the cAu-TNF vectors without derivatized PEG, injection of the PT-cAu-TNF vector resulted in elevated levels of TNF in the circulation throughout the time course studied. Six hours after injection with native TNF, the TNF levels were only 2% of that seen at 5 minutes (FIG. 5B). In contrast, mice receiving the PT-cAu-TNF vector had TNF blood levels which were approximately 30% of their maximal 5-minute values. At this 6-hour time point, blood TNF levels in mice treated with the PT-cAu-TNF formulation were 23-fold higher than those in mice treated with native TNF.

FIG. 5B shows the TNF pharmacokinetic analysis. Mice were bled through the retro-orbital sinus at 5, 180 and 360 minutes after the injection. The blood samples were centrifuged at 14,000 rpms and the resultant serum analyzed for TNF concentration using an EIA. Data are presented as the mean±SEM serum TNF concentration from 3 mice/time point. (*p<0.05).

In those animals treated with PT-cAu-TNF vector, TNF accumulated in the tumor. As shown in FIG. 5C, the maximal intra-tumor concentration of TNF observed in those mice treated with native TNF was 0.8 ng of TNF/mg protein. The peak amount was seen within five minutes of administration of the native TNF, and did not increase over the 6-hours. In contrast, those animals treated with PT-cAu-TNF vector had intra-tumor levels of TNF that increased over time. TNF was actively sequestered in the tumor of those animals treated with PT-cAu-TNF vector. By the end of the time period, nearly 10-times more TNF was found in the tumors of those animals treated with the PT-cAu-TNF vector compared to those treated with native TNF or the 4 μg/ml cAu-TNF vector (FIG. 5D).

In FIG. 5C, the intra-tumor TNF distribution over time is shown. Mice were sacrificed 5, 180 and 360 minutes after the injection of 15 micrograms of native TNF or PT-cAu-TNF vector. The tumors were removed and analyzed for TNF and total protein. Data are presented as the mean±SEM of tumor TNF concentration, expressed in ng TNF/mg of total protein, from 3 mice/time point/treatment group. (Δp<0.1, *p<0.05).

FIG. 5D shows a comparison of the intra-tumor TNF concentrations from animals injected intravenously with 15 micrograms of either the 4 μg/ml cAu-TNF vector or the PT-cAu-TNF vector.

The accumulation of the PT-cAu-TNF vector in the MC-38 tumor mass was not a passive event or just a function of the vector's extended residency time in the circulation since, over the same period of time, TNF did not accumulate in other organs, such as the lung, liver, and brain. Rather, the presence of TNF in these organs was similar in pattern to that seen in blood. Furthermore, the distribution of the drug in these non-targeted organs was similar to that seen with native TNF. Consequently, the accumulation of TNF resulting from the administration of the PT-cAu-TNF vector is specific to the tumor (FIGS. 5 E and F).

FIGS. 5 E and F show the distribution of TNF in various organs from MC-38 tumor-burdened C57/BL6 mice receiving either native TNF (FIG. 5E) or PT-cAu-TNF (FIG. 5F). Livers, lung and brains from the animals treated in this Example were processed and analyzed for TNF and protein concentrations. Data are presented as the mean±SEM of intra-organ TNF concentration from 3 mice/time point/formulation injected.

### Example 11

### Dose Escalation, Toxicity and Efficacy

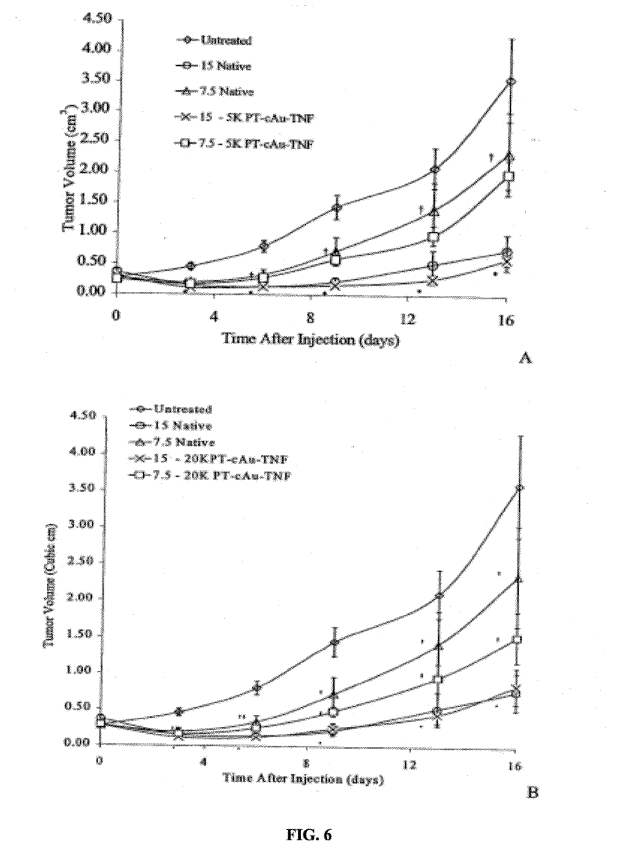

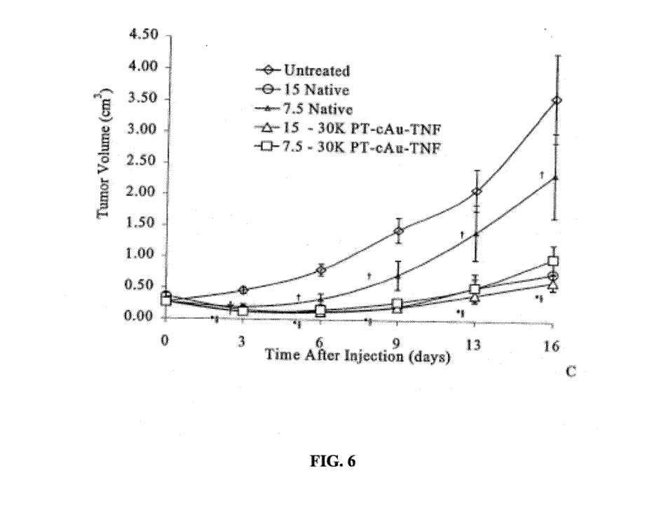

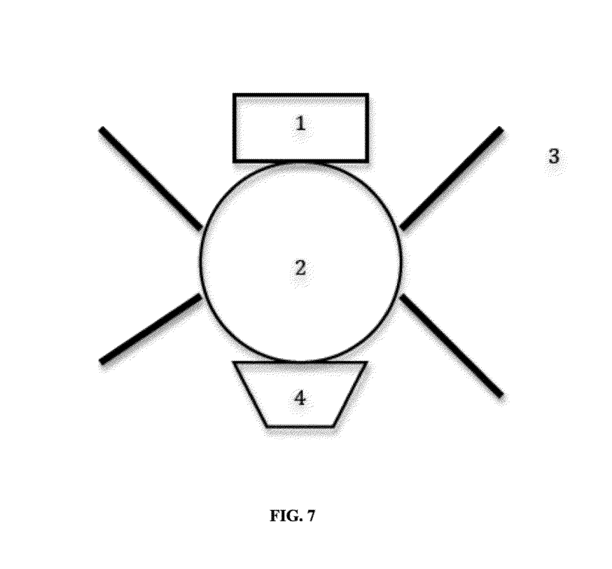

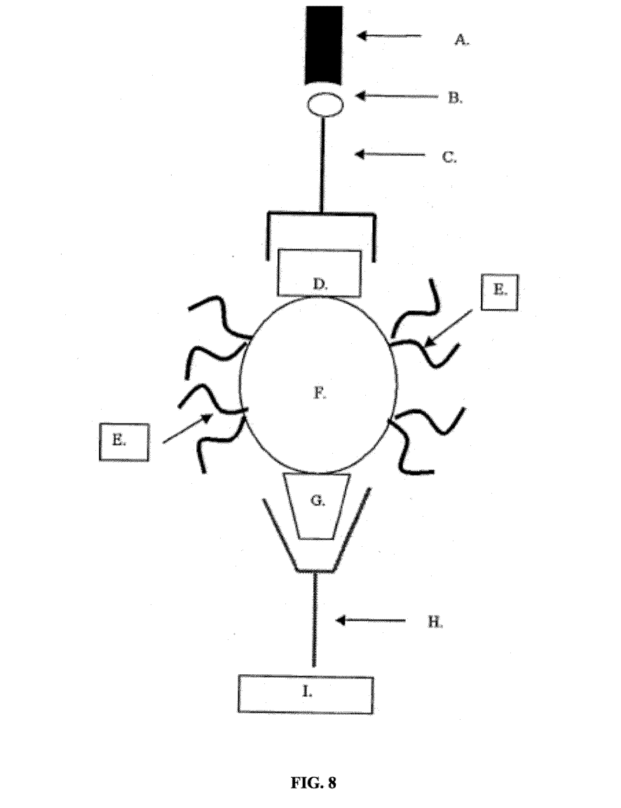

C57/BL6 mice were implanted with the colon carcinoma cell line, MC-38, as a model to compare the safety and efficacy of native and colloidal gold bound TNF preparations. C57/BL6 mice were implanted with 10⁵ MC-38 tumor cells in one site on the ventral surface. The cells were allowed to grow until they formed a tumor measuring 0.5 cm³ as determined by measuring the tumor in three dimensions (L×W×H). MC-38 tumor burdened C57/BL6 mice (n=4-9/group) were intravenously injected with increasing doses of native TNF, cAu-TNF vector, or PT-cAu-TNF vector. The mice were divided into nine groups with 4-9 animals/group. One group served as an untreated control group. Two groups were intravenously injected with either 7.5 or 15 micrograms of native TNF, (FIG. 6A). Two groups were intravenously injected with either 7.5 or 15 micrograms of a 20 K-PT-cAu-TNF vector (FIG. 6B). Two groups were intravenously injected with either 7.5 or 15 micrograms of the 30 K-PT-cAu-TNF vector (FIG. 6C). Tumor measurements were made on various days after the treatment on animals that survived TNF treatment. Statistical difference between the various groups was determined using a paired t-test.

The mice were scored at different times after injection using the following toxicity rating scale: 0=normal activity; 1=piloerection; 2=loose stools; 3=lethargy; 4=unresponsive; and 5=death. Animals scoring a 4 on two consecutive scoring times were sacrificed. Treatment efficacy was determined by monitoring the reduction in tumor volume induced by the various TNF treatments. The measures were compared to the initial tumor volume of each animal in the various groups as well as animals receiving saline injections (untreated controls). Untreated controls were sacrificed when their tumor volumes were 4 cm³.

A 5K-PT-cAu-TNF vector, comprising PEG-thiol of molecular weight 5,000 daltons, was tested for safety and efficacy in dose escalation studies in MC-38 tumor-burdened mice. Like the 4 μg/ml cAu-TNF vector, the 5K-PT-cAu-TNF vector had an improved safety profile when compared to native TNF. At a dose of 15 micrograms of native TNF/mouse, 33% (3 out of 9) of the animals died within 24 hours of treatment. In addition, 7.5 micrograms of native TNF resulted in 1 out of the 9 animals dying. In contrast, none of the animals receiving either 7.5 or 15 micrograms of TNF bound to the 5K-PT-cAu-TNF vector died. These vector-treated animals exhibited only transient adverse clinical effects. No appreciable difference in the anti-tumor efficacy of the 5K PT-cAu-TNF vector, compared to native TNF, was observed following a single treatment (FIG. 6A). These findings were replicated in two experiments.

FIG. 6A is a graph comparing safety and efficacy of native TNF or PT-cAu-TNF vectors. † p<0.05 for the 7.5 micrograms of dose of native TNF or PT-cAu-TNF treatment versus untreated controls. * p<0.05 for the 15 micrograms of dose of native or PT-cAu-TNF treatment versus untreated controls and 7.5 micrograms of dose native and PT-cAu-TNF.

The effect of PEG-thiol chain length on vector anti-tumor efficacy is shown in FIGS. 6 B-C. At the highest TNF dose (15 micrograms) no differences were noted among any of the TNF vectors compared to native TNF. However, at a lower dose of 7.5 micrograms of TNF, a pattern emerged. As noted above, 5K PEG-thiol did not markedly improve the anti-tumor effect of the colloidal gold vector compared to native TNF. By increasing the PEG chain length to 20K there was a slight, non-statistical improvement in tumor reduction (FIG. 6B), while increasing it to 30K resulted in a marked, statistically significant improvement in tumor regression (FIG. 6C). With the 30K PT-cAu-TNF vector, animals treated with a dose of 7.5 micrograms of TNF via this vector, had residual tumors of similar size to those treated with 15 micrograms native TNF. In contrast to those treated with 15 micrograms of native TNF, the 30K PT-cAu-TNF-treated animals administered 7.5 micrograms of TNF experience no toxicity. In effect, a single injection of a 30K PT-cAu-TNF vector which gave less TNF but induced the same maximal anti-tumor regression as that seen with twice as much native TNF, and the treated subject survived the treatment. In this tumor model, a single injection of either 5K or 20K PEG-Thiol-cAu-TNF vector was safer than native TNF, and the 30K PEG-Thiol-cAu-TNF vector was both safer and more efficacious than the native molecule.

FIG. 6B is a graph comparing native TNF and 20 K-PT-cAU-TNF safety and efficacy. †,§ p<0.05 for the 7.5 micrograms of dose of native TNF or PT-cAu-TNF vector treatment, respectively, versus untreated controls. 7.5 micrograms of the 20 K-PT-cAu-TNF vector was not statistically different form the 7.5 micrograms native group*p<0.05 for the 15 micrograms of dose of native or PT-cAu-TNF treatment versus untreated controls and the 7.5 micrograms of native and 20 K-PT-cAu-TNF vector.

FIG. 6C is a graph comparing native TNF and 30 K-PT-cAU-TNF safety and efficacy. † p<0.05 for the 7.5 micrograms of dose of native TNF versus untreated controls. § p<0.05 for the 7.5 micrograms of dose of the 30 K-PT-cAu-TNF vector treatment versus untreated controls and native TNF groups. * p<0.05 for the 15 micrograms of dose of native or PT-cAu-TNF vector treatment versus untreated controls and the 7.5 micrograms of dose native TNF. 7.5 micrograms of the 30 K-PT-cAu-TNF was not statistically different from 15 micrograms of native or 30 K-PT-cAu-TNF.

### Example 12

### Oral Administration of Colloidal Gold Compositions

The effect of the route of administration on the tumor sequestration of PT-cAu-TNF vectors was tested. The vector preparation is as described in previous Examples. Briefly colloidal gold is bound to TNF at a concentration of 0.5 micrograms/ml using the in-line mixing apparatus described above. Following a 15-minute incubation, 30,000 daltons PEG-thiol (dissolved in pH 8 water) is added to the mixture at a final concentration of 12.5 micrograms/ml. The solution is stirred and immediately processed by diafiltration. The retentate is sterile filtered and aliquoted for storage at −40° C.

MC38 tumor-burdened C57/BL/6 mice were used as a model to determine the ability of orally administered PT-cAu-TNF vector to target the delivery of TNF and gold to the tumor site. For these studies mice (n=3) were anesthetized with 1 mg of pentobarbitol. After the animals were completely sedated, 26 micrograms of TNF bound onto the PT-cAu-TNF vector was administered through an oral cannula. The animals were allowed to recover and were allowed free access to food and water. The following day, it was observed that the tumors were the familiar red/blue color of the colloidal gold vector. These data show oral administration of the PT-cAu-TNF vector can be used to treat tumors.

### Example 13

### In Vitro Activity of Colloidal Bound TNF Vectors

The in vitro activity of native TNF and the cAu-TNF vectors were determined by the WEHI-164 TNF bioassay as described by Khabar, K. S., Siddiqui, S., and Armstrong, J. A., WEHI-13 VAR: a stable and sensitive variant of WEHI 164 clone 13 fibrosarcoma for tumor necrosis factor bioassay, Immunol. Lett 46: 107-110 (1995). In this bioassay, 10⁴ WEHI-164 cells were plated in 12-well tissue culture clusters. The cells were cultured in DMEM supplemented with 10% FBS. Native TNF and four different cAU-TNF vectors prepared at 0.5, 1, 2 and 4 micrograms of TNF/ml of colloidal gold were incubated for 7 days with the cells at final TNF concentrations ranging from 1 mg/ml to 0.0001 mg/ml. Cell number was determined on day 7 using a Coulter Counter. Data are presented as the mean±SEM of the cell number for triplicate wells/TNF formulation.

The cAu-TNF vectors were biologically equivalent on a molar basis to native TNF in the WEHI 164 bioassay. For example, 12.5 ng of native TNF inhibited WEHI 164 cell growth by 50%, whereas the same 12.5 ng dose of the 1.0, 2.0 and 4.0 micrograms/ml cAu-TNF preparations inhibited WEHI 164 cell growth by 47%, 55%, and 52%, respectively.

### Example 14

### PEG-Thiol Vector for Tumor-Targeted Delivery of Anti-Angiogenic Drugs

These experiments used a PT-cAU-TNF-endostatin vector, a vector comprising two agents. It is thought that the TNF provided targeting functions for delivery of the therapeutic agent, endostatin (END), to the tumor. It is also theorized that once at the target, both agents may provide therapeutic effects. An aspect of the vector composition is the ratio of the targeting molecule, the therapeutic molecule and the PEG. All three entities are found on the same particle of colloidal gold. A schematic of this vector is shown in FIG. 7. In FIG. 7, 1=an agent, such as an END molecule; 2=a colloidal gold particle; 3=derivatized PEG; and 4=a different agent or targeting molecule, such as a TNF molecule.

The PT-cAu_{(TNF)}-END vector, comprising derivatized PEG, TNF and endostatin (END) associated with a colloidal gold particle, was made using the apparatus described in FIG. 1. The PT-cAu_{(TNF)}-END was made in three steps. First, TNF associated with the gold particles at a very low subsaturating mass of TNF. Unlike the PT-cAu-TNF vector, which was made with a concentration of TNF of 0.5 micrograms/ml, this vector was made with a TNF concentration of 0.05 micrograms/ml. TNF (diluted in 3 mM CAPS buffer, pH=10) which was added to the reagent bottle of the apparatus at a concentration of 0.1 micrograms/ml. The second bottle in the apparatus was filled with an equal volume of colloidal gold at a pH of 10. TNF was bound to the colloidal gold particles by activation of the peristaltic pump as previously described. The colloidal gold-TNF solution was incubated for 15 minutes and subsequently placed back into the gold container of the apparatus. The reagent bottle was then filled with an equal volume of endostatin (diluted in CAPS buffer at a concentration of 0.15 to 0.3 micrograms/ml. In an alternative embodiment, endostatin may be chemically modified by the addition of a sulfur group using agents such as n-succinimidyl-5-acetylthioacetate, to aid in binding to the gold particle.

The peristaltic pump was activated to draw the colloidal gold bound TNF and endostatin solutions into the T-connector. Upon complete interactions of the solutions the mixture was incubated in the collection bottle for an additional 15 minutes. The presence of additional binding sites for the PEG-Thiol was confirmed by the ability of salt to precipitate the particle at this stage. After the 15 minute incubation, 5K PEG-Thiol was added to the cAu_{(TNF)}-END vector and concentrated by diafiltration as previously described.

An alternative method for binding the two proteins to the same particle of gold comprising using the same apparatus as FIG. 1 and adding the agents simultaneously to the gold. TNF and END were placed in the reagent chamber of the binding apparatus. The concentration of each protein was 0.25 micrograms/ml and as a result, 1 ml of solution contained 0.5 micrograms of total protein. After binding the dual agent composition to gold particles, this colloidal gold preparation also precipitated in the presence of salt, indicating that additional free binding sites were available to bind the PEG-thiol. After a 15 minute incubation, 5K PEG-Thiol was added to the cAu_{(TNF)}-END vector and subsequently processed as described above.

After diafiltration, the retentate was measured for TNF and END concentrations in their respective EIA. To confirm the presence of END and TNF on the same particle of colloidal gold, a cross-antibody capture and detection assay was designed and used. A schematic representation of this EIA is shown in FIG. 8. In FIG. 8, A=a labeled binding partner, such as streptavidin alkaline phosphatase; B=the binding partner, such as biotin; C=detection antibody, such as biotinylated anti-END antibody; D=an agent, such as an END molecule; F=a colloidal gold particle; E=derivatized PEG; and G=a different agent or targeting molecule, such as a TNF molecule; H=a capture antibody; such as anti-TNF antibody; and L=a support, such as a bead or a microtiter plate.

Samples of the PT-cAu_{(TNF)}-END vector were added to EIA plates coated with either the TNF or END capturing antibodies. The samples were incubated with the capturing antibody for 3 hours. After incubation the plates were washed and blotted dry. To bind any END present on a TNF captured sample, a biotinylated rabbit anti-endostatin polyclonal antibody was added to the wells. After a 30-minute incubation, the plates were washed and the presence of the biotinylated antibody was detected with streptavidin conjugated alkaline phosphatase. The generation of a positive color signal by the endostatin detection system indicated that the detection antibody bound to the chimeric vector previously captured by the TNF monoclonal antibody. See FIG. 9. By reversing the capturing and detection antibodies and using appropriate secondary detection systems, an assay was used to detect the presence of TNF on an END-captured particle. See FIG. 9. FIG. 9 is a graph showing TNF- and END-captured vectors exhibiting the presence of the second agent.

The data from these studies are presented in Table VI. As can be seen in Table VI, the retentate of the vector samples had 17 μg/ml of TNF and 22 μg/ml of END. These same samples also generated positive signals in the cross-antibody assays suggesting that both TNF and endostatin were on the same particle of colloidal gold (FIG. 9).

TABLE VI
The TNF and Endostatin concentrations
present in retentates of the PT-cAU_{(TNF)}-END vectoc.

| Sample | Analyte Tested | Concentration |
|---|---|---|
| PT-cAU_{(TNF)}-END | TNF | 17 μg/ml |
| | END | 22 μg/ml |

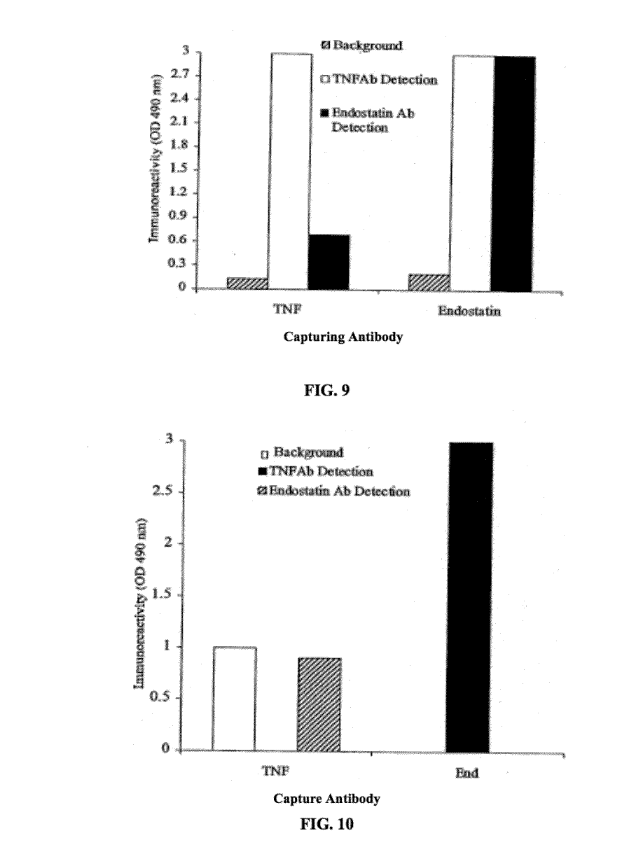

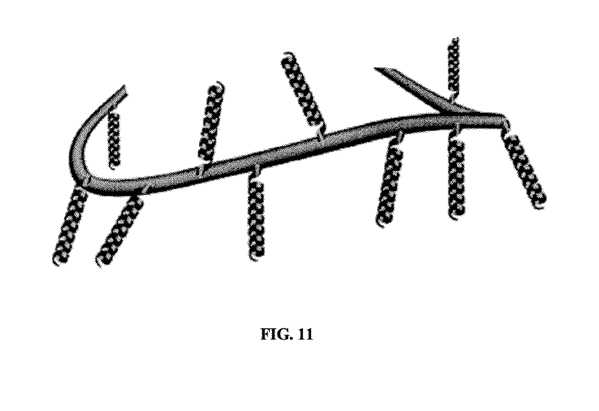

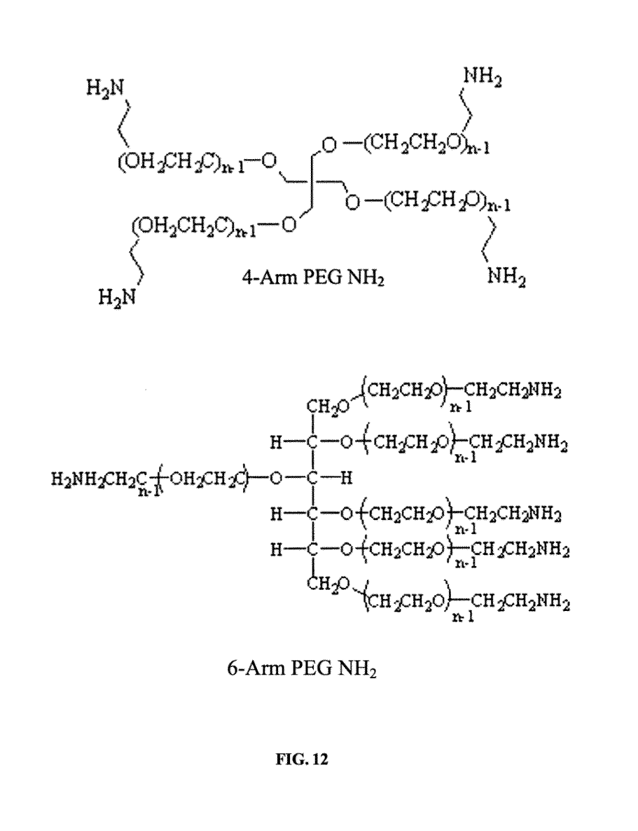

In FIG. 10 are the data showing the detection of endostatin and TNF from the PT-cAu_{(TNF)}-END vector in resected MC-38 tumors following intravenous injection. These data show that the PT-cAu(TNF)-END vector reached the tumor without degradation, since both molecules were detected in the tumor tissue.

### Example 15

### Binding of Branched Aminated PEG to Colloidal Particles

To demonstrate the binding of branched aminated PEG to the colloidal gold nanoparticles increasing amounts of either 4- or 6-arm PEGNH₂ (see FIG. 16 for general structures) of varying MW were incubated with 27 nm colloidal gold nanoparticles. The 4-arm PEGNH₂ variants were synthesized (SunBio, Inc.) to an average MW of either 10 or 20 kD, while the two 6-arm PEGNH₂ preparations had an average MW of 15 and 20 kD.

Typically colloidal gold nanoparticles maintain their colloidal state by mutual electrostatic repulsion generated and maintained by a net negative charge on their surface. Cations present in salt solutions negate this charge repulsion and cause the unmodified particles to agglomerate and eventually precipitate out of solution. Salt-induced precipitation is easily tracked as the particles undergo a gradual change in color from orange/red (the color of mono-dispersed smaller particles) to brownish/purple (the color of mono-dispersed larger particles) to purple/gray (the color of early particle agglomeration), and finally to black (the color of gold nanoparticle aggregates). These aggregates interact and ultimately precipitate out of solution. Binding of proteins (see previous patent) and polymers, such as the aminated PEGs, to the particles' surface maintains the particles in a colloidal state by blocking the salt-induced precipitation of the colloidal gold particles.

To demonstrate the binding of the aminated PEGs to colloidal gold nanoparticles a saturation study was conducted. For this study the various PEGN₂ preparations were diluted in DIH₂O to a stock concentration of 10 mg/mL and further diluted using a 0.1 M NaHCO₃ buffer to a working solution of 1 mg/mL. Subsequently, increasing amounts (0.625 μg to 10 μg) of the various PEGNH₂ were added to designated 1 mL aliquots of the 27 nm colloidal gold particles. The samples were incubated for 30 minutes at room temperature for 30 minutes. To demonstrate the ability of the various PEGNH₂ to bind and stabilize the colloidal gold nanoparticle 100 μL of a 10% NaCl solution was added to each sample.

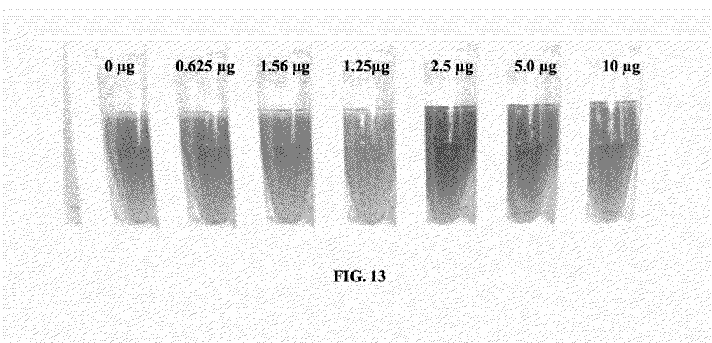

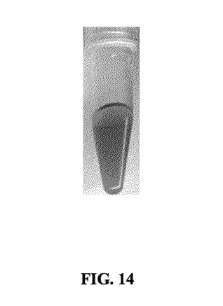

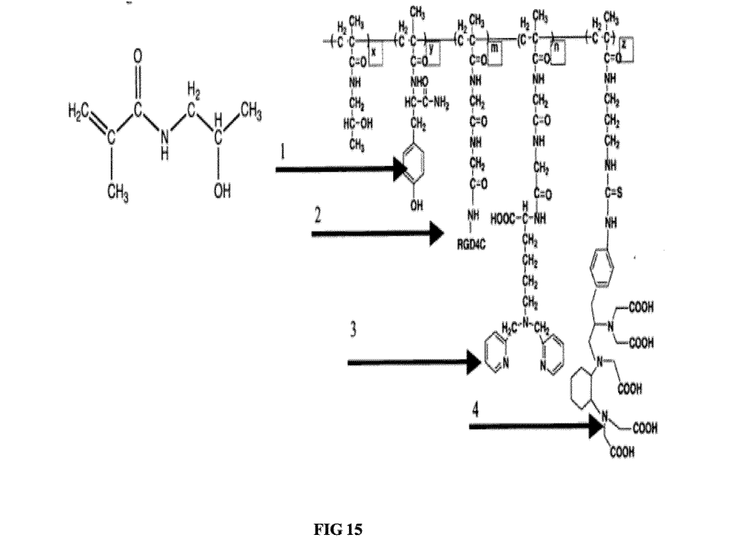

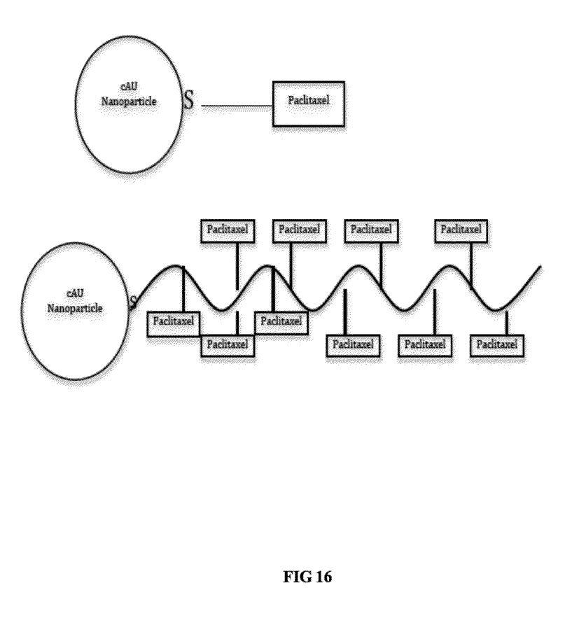

The data shown in FIG. 13 demonstrate the resultant saturation binding of the PEGNH₂ to the gold nanoparticles. At low doses the PEGNH₂ sparingly coated the surface of the nanoparticles, resulting in poorly stabilized particles when exposed to salt-induced precipitation. This change in particle dispersity was evidenced by the change in the color, as seen in FIG. 13, where the sols were no longer red (i.e., mono-dispersed state), but rather they became purple and black as the particles agglomerated (purple) and precipitated. In contrast, at higher concentrations the PEGNH₂ completely covered the surface of the gold nanoparticles, preventing the salt-induced precipitation and keeping the particles in a mono-dispersed state.

At low doses, 0.625 μg to 1.25 μg of the aminated PEG sparingly coated the surface of the 27 nm colloidal gold nanoparticles. Consequently, when exposed to salt the particles precipate as evidenced by the color change of the sol from red to purple and ultimately to black. At higher doses, the 10 kD PEG amine completely covered the surface of the particles keeping the nanoparticles in a stable mono-dispersed colloidal state, as noted by the red color of the sols in the presence of salt. Similar results were observed with all of the various aminated PEG preparations.

To demonstrate that the PEGNH₂ was actually bound to the gold nanoparticle surface a second study was conducted in which 10 μg of the 10 kD 4-arm PEGNH₂ was added to the gold nanoparticles and incubated as described above. Following this incubation, the solution was centrifuged and the supernatant was removed. The colloidal gold nanoparticle pellet was resuspended in 1 mL of DIH₂O. The sample was sonicated and 100 μL of 10% NaCl was added to the sol to induce particle precipitation. As shown in FIG. 3 the color of the sol remained red, supporting the conclusion that the PEGN₂ was indeed bound to the gold nanoparticles. If the PEGNH₂ was simply a stabilizer, then centrifugation should have removed it and with the addition of salt the solution should have changed to purple and then black. As seen below, the particles remained mono-dispersed (i.e., did not undergo agglomeration and precipitation) as evidenced by the red color of the reconstituted nanoparticles.

After incubating 27 nm colloidal gold nanoparticles with 10 μg of the 10 kD 4-arm PEGNH₂, samples were centrifuged to separate the particles from unbound polymer. The supernatant was removed and the colloidal gold nanoparticles were re-suspended in DIH₂O and sonicated. Following re-suspension, a NaCl (10% v/v) solution was added to induce particle precipitation. By preventing salt-induce precipitation, the data demonstrate that after centrifugation the PEGNH₂ was bound to the colloidal gold nanoparticles.

It must be noted that, as used in this specification and the claims, the singular forms “a,” “an,” and “the” include plural referents unless the context clearly dictates otherwise. Thus, for example, reference to a vector composition containing “an agent” means molar quantities of such an agent.

It is to be understood that this invention is not limited to the particular combinations, methods, and materials disclosed herein as such combinations, methods, and materials may vary somewhat. It is also to be understood that the terminology employed herein is used for the purpose of describing particular embodiments only and is not intended to be limiting.

We claim:

1. A nanotherapeutic vector composition, comprising a nanoparticle platform, a stealth agent and one more more active agents, wherein the stealth agent and one or more active agents are bound to the nanoparticle platform.

2. The nanotherapeutic vector composition of claim 1, wherein the nanoparticle platform is selected from the group consisting of; a colloidal metal, a nanoshell, a nanorod, a quantum dot, a nanocluster, a liposome, a dendrimer, a metal-liposome particle, a metal-dendrimer nanohybrid, and a nanotube.

3. The nanotherapeutic vector composition of claim 2, wherein the nanoparticle platform is a colloidal metal and wherein the colloidal metal comprises a material selected from the group consisting of; gold, silver, silica, and iron.

4. The nanotherpeutic vector composition of claim 3, wherein the colloidal metal is colloidal gold.

5. The nanotherapeutic vector composition of claim 1, wherein the stealth agent is selected from the group consisting of; polyethylene glycol (PEG), hyroxyethyl starch (HES/HAES), PolyPEG®, rPEG, branched aminated PEGs, polyoxypropylene polymers, block or triblock copolymers comprising polyoxyethylene/polyoxypropylene/polyoxyethylene blocks, and methacrylamide polymers.

6. The nanotherapeutic vector composition of claim 5, wherein the stealth agent is a branched aminated PEG.

7. The nanotherapeutic vector composition of claim 6, wherein the branched aminated PEG is a 4 arm or 6 arm branched aminated PEG.

8. The nanotherapeutic vector composition of claim 5, wherein the stealth agent is a methacrylamide polymer.

9. The nanotherapeutic vector composition of claim 8, wherein the methacrylamide polymer is a N-(2-hydroxypropyl)methacrylamide polymer.

10. The vector composition of claim 1, wherein the one or more active agents is tumor necrosis factor alpha (TNF) and paclitaxel.

11. The vector composition of claim 1, further comprising a targeting ligand.

12. A nanotherapeutic vector composition comprising one or more functionalized polymers attached to a nanoparticle platform, wherein the functionalized polymer comprises one or more active agents attached to a polymer backbone.

13. The nanotherapeutic vector composition of claim 12, wherein the functionalized polymer comprises a methacrylamide backbone.

14. The nanotherapeutic vector composition of claim 13, wherein the methacrylamide backbone comprises N-(2-hydroxypropyl)methacrylamide monomers, wherein a therapeutic agent, diagnostic agent, or targeting ligand is attached to each monomer.

15. The nanotherapeutic vector composition of claim 14, wherein paclitaxel is attached the methacrylamide backbone.

16. The nanotherapeutic vector composition of claim 14, wherein paclitaxel and TNF are attached to the methacrylamide backbone.

17. The nanotherapeutic vector composition of claim 14, wherein a diagnostic agent is attached to the methacrylamide backbone.

18. The nanotherapeutic vector composition of claim 17, wherein the diagnostic agent is chelating agent for an imaging radionuclide.

19. The nanotherapeutic vector composition of claim 18, wherein the chelating agent is ^{99m}Tc.

20. The nanotherapeutic vector composition of claim 14, wherein a chelating agent for a therapeutic nucleotide is attached to the methacrylamide backbone.

21. The nanotherapeutic vector composition of claim 18, wherein the therapeutic radionuclide is ⁹⁰Y.

22. The nanotherapeutic vector composition of claim 14, wherein the functionalized polymer further comprises a stealth domain comprising unmodified methacrylamide monomers.
